(19) United States
(12) Patent Application Publication
(10) Pub. No.: US 2026/0338729 A1
(43) Pub. Date: Oct. 29, 2026

(54) GAS SENSOR CHIP, GAS SENSING SYSTEM, AND PRODUCTION METHOD FOR GAS SENSOR CHIP

(71) Applicant: Hitachi, Ltd., Tokyo (JP)

(72) Inventors: Yoshitaka SASAGO, Tokyo (JP); Hiroyuki UCHIYAMA, Tokyo (JP); Yuan BU, Tokyo (JP); Takayasu NORIMATSU, Tokyo (JP)

(73) Assignee: Hitachi, Ltd., Tokyo (JP)

(21) Appl. No.: 19/110,899

(22) PCT Filed: Sep. 8, 2023
(86) PCT No.: PCT/JP2023/032889

§ 371 Date: Mar. 12, 2025

(30) Foreign Application Priority Data
Nov. 14, 2022 (JP) 2022-182007

(51) Int. Cl. G01N 27/414; G01N 27/416; G01N 33/00

(52) U.S. Cl. CPC G01N 27/4141; G01N 27/4162; G01N 33/0027

(57) ABSTRACT

Sensing of plural types of gases present in the atmosphere is realized at a low cost. A gas sensor chip has a semiconductor substrate and a plurality of gas detection material layers formed at respectively different positions on the semiconductor substrate. The gas detection material layers each have a metal oxide layer and a catalyst metal layer formed over the metal oxide layer and have a structure in which the catalyst metal layer is exposed to the atmosphere. The gas detection material layers are different from each other in the combination of the type, film thickness, and crystal particle size of the material of the metal oxide layer and the type, film thickness, and crystal particle size of the material of the catalyst metal layer.

### TECHNICAL FIELD

This invention relates to a gas sensor chip, a gas sensing system, and a production method for the gas sensor chip.

### BACKGROUND ART

Power generation technologies using hydrogen or carbon-neutral biofuel instead of fossil fuel are attracting attentions to achieve a decarbonized society. Since awareness for an environment is growing among people all over the world, an interest in pollutant components contained in the atmosphere is also increasing. With an increasing interest in well-being, people monitor their health condition, for example, by breath sensing and want to live in a comfortable environment. As a result, in addition to an increasing need for sensing of a single gas such as hydrogen, there is an increasing need for detecting the respective concentrations of gas components in the atmosphere containing plural types of gases or separating or discriminating the combination pattern of the plural types of gas components.

Patent Literature 1 describes the technology of a gas sensor having an array comprised of a gas molecule detection unit and an amplifier circuit.

Non-Patent Literature 1 describes a hydrogen sensor which is one of gas sensors. As a hydrogen sensor, there are some sensors different in detection principle. Of these, FET type, capacitor type, and diode type sensors are classified into a work function type sensor.

### CITATION LIST

### Patent Literature

Patent Literature 1: WO No. 2017/104130

### Non-Patent Literature

Non-Patent Literature 1: Sensors and Actuators B 157 (2011) 329-352

### SUMMARY OF INVENTION

### Technical Problem

For detecting the type of a gas or a component pattern of each gas present in the atmosphere, plural types of gas sensor elements different in characteristics depending on the gas type or the gas concentration become necessary. Manufacturing such plural types of gas sensor elements individually or getting them individually generally leads to a high cost.

Patent Literature 1 and Non-Patent Literature 1 however have therein no description on the technology of manufacturing the aforesaid plural types of gas sensor elements at a low cost and it is difficult to achieve sensing of plural types of gases present in the atmosphere at low cost.

Under such situations, there is therefore a demand for the development of a technology capable of achieving sensing of plural types of gases present in the atmosphere at low cost.

### Solution to Problem

As a means for overcoming the aforesaid problem, the technology described in Claims is used. As one example of the technology, a gas sensor chip may be used which includes: a semiconductor substrate; and a plurality of gas detection material layers formed respectively at different positions on the semiconductor substrate. The gas detection material layers are configured to have a metal oxide layer and a catalyst metal layer formed over the metal oxide layer, the catalyst metal layer being exposed to the atmosphere. The gas detection material layers are different from each other in the combination of the type, film thickness, and crystal particle size of material of the metal oxide layer and the type, film thickness, and crystal particle size of material of the catalyst metal layer.

In addition, as another example, a gas sensing system is used which includes: a gas sensor chip; a heater unit that heats the gas sensor chip to elevate a temperature thereof; and a control unit that controls the gas sensor chip and the heater unit. The gas sensor chip includes a semiconductor substrate and a plurality of gas detection material layers formed at respectively different positions on the semiconductor substrate. The gas detection material layers are configured to have a metal oxide layer and a catalyst metal layer formed over the metal oxide layer, the catalyst metal layer being exposed to the atmosphere. The gas detection material layers are different from each other in a combination of a type, a film thickness, and a crystal particle size of the metal oxide layer and a type, a film thickness, and a crystal particle size of the catalyst metal layer. The control unit controls the heater unit so that the gas sensor chip has a predetermined temperature and detects a gas in the atmosphere based on information obtained using the gas detection material layers.

Further, as further example, a method of producing a gas sensor chip is used which includes: a step of forming an impurity layer on a semiconductor substrate; a step of forming a first gas detection material layer including a catalyst metal layer on the semiconductor substrate; and a step of forming a second gas detection material layer containing a catalyst metal layer at a position different from that of the first gas detection material layer, on the semiconductor substrate. The first gas detection material layer is different from the second gas detection material layer in at least one of a type, a film thickness, and a crystal particle size of a material constituting the gas detection material layers.

### Advantageous Effects of Invention

The present invention makes it possible to achieve sensing of plural types of gases present in the atmosphere at low cost. Problems, constitution, and effect other than those described above will be apparent from the following description of the embodiments.

### BRIEF DESCRIPTION OF THE DRAWINGS

FIG. 1A shows one example of a gas sensing system of First Embodiment.

FIG. 1B shows one example of a hardware constitution of the gas sensing system of First Embodiment.

FIG. 2 is a plan view of the gas sensor chip of First Embodiment.

FIG. 3A schematically shows one example of the cross-section of a sensor FET (SFET).

FIG. 3B schematically shows one example of the cross-section of a reference FET (RFET).

FIG. 4 shows one example of a material used for a gas detection material layer of the sensor FET and the reference FET.

FIG. 5 shows one example of the operation during detection of the concentration of a detection target gas in a gas sensor chip using an FET type sensor.

FIG. 6A shows gate voltage—drain current characteristics in an NFET type sensor FET.

FIG. 6B shows gate voltage—drain current characteristics in an NFET type reference FET.

FIG. 7 shows one example of the relationship between a gas concentration and a gate threshold voltage shift in a sensor FET.

FIG. 8A shows one example of how two sensor FETs respond to some gases having a certain concentration.

FIG. 8B is a view for describing that the concentration of M types of gases can be estimated using N pieces of sensor FETs different from each other.

FIG. 9 shows an example of comparison results of the size and cost per gas of the gas sensor between the gas sensor of First Embodiment and a conventional gas sensor.

FIG. 10 shows the first example of an FET type gas sensor array using a selection transistor.

FIG. 11 shows the second example of an FET type gas sensor array using a selection transistor.

FIG. 12 shows the third example of an FET type gas sensor array using a selection transistor.

FIG. 13 shows one example of an FET type gas sensor array using TSV.

FIG. 14 shows one example of an FET type gas sensor array using an interposer.

FIG. 15A shows gate voltage—drain current characteristics in a PFET type sensor FET.

FIG. 15B shows gate voltage—drain current characteristics in a PFET type reference FET.

FIG. 16 shows the first example of a device that determines a gas concentration by using an NFET type sensor FET and a PFET type sensor FET in combination.

FIG. 17 shows the second example of a device that determines a gas concentration by using an NFET type sensor FET and a PFET type sensor FET in combination.

FIG. 18A schematically shows one example of the cross-section of a sensor capacitor.

FIG. 18B schematically shows one example of the cross-section of a reference capacitor.

FIG. 19 shows one example of the operation during detection of the concentration of a detection target gas in a gas sensor chip using a capacitor type sensor.

FIG. 20A shows one example of an electrostatic capacity—gate voltage characteristic curve of a sensor capacitor.

FIG. 20B shows one example of an electrostatic capacity—gate voltage characteristic curve of a reference capacitor.

FIG. 21A schematically shows one example of the cross-section of a sensor diode.

FIG. 21B schematically shows one example of the cross-section of a reference diode.

FIG. 22 shows one example of the operation during detection of the concentration of a detection target gas in a gas sensor chip using a diode type sensor.

FIG. 23A shows one example of a current—voltage characteristic curve of a sensor diode.

FIG. 23B shows one example of a current—voltage characteristic curve of a reference diode.

FIG. 24A is a view for describing one example of a production method of the gas sensor chip of First Embodiment.

FIG. 24B is another view for describing one example of the production method of the gas sensor chip of First Embodiment.

FIG. 24C is a further view for describing one example of the production method of the gas sensor chip of First Embodiment.

FIG. 24D is a still further view for describing one example of the production method of the gas sensor chip of First Embodiment.

FIG. 24E is a still further view for describing one example of the production method of the gas sensor chip of First Embodiment.

FIG. 24F is a still further view for describing one example of the production method of the gas sensor chip of First Embodiment.

FIG. 25 is a flow chart showing one example of a production method of the gas sensor chip of First Embodiment.

FIG. 26A is a view for describing Modification Example 1 of the production method of the gas sensor chip.

FIG. 26B is another view for describing Modification Example 1 of the production method of the gas sensor chip.

FIG. 26C is a further view for describing Modification Example 1 of the production method of the gas sensor chip.

FIG. 26D is a still further view for describing Modification Example 1 of the production method of the gas sensor chip.

FIG. 27 is a flow chart showing Modification Example 1 of the production method of the gas sensor chip.

FIG. 28A is a view for describing Modification Example 2 of the production method of the gas sensor chip.

FIG. 28B is another view for describing Modification Example 2 of the production method of the gas sensor chip.

FIG. 28C is a further view for describing Modification Example 2 of the production method of the gas sensor chip.

FIG. 28D is a still further view for describing Modification Example 2 of the production method of the gas sensor chip.

FIG. 29A is a view for describing Modification Example 3 of the production method of the gas sensor chip.

FIG. 29B is another view for describing Modification Example 3 of the production method of the gas sensor chip.

FIG. 29C is a further view for describing Modification Example 3 of the production method of the gas sensor chip.

FIG. 29D is a still further view for describing Modification Example 3 of the production method of the gas sensor chip.

FIG. 30A is a view for describing Modification Example 4 of the production method of the gas sensor chip.

FIG. 30B is another view for describing Modification Example 4 of the production method of the gas sensor chip.

FIG. 30C is a further view for describing Modification Example 4 of the production method of the gas sensor chip.

FIG. 30D is a still further view for describing Modification Example 4 of the production method of the gas sensor chip.

FIG. 30E is a still further view for describing Modification Example 4 of the production method of the gas sensor chip.

FIG. 30F is a still further view for describing Modification Example 4 of the production method of the gas sensor chip.

FIG. 31 is a view showing an example of a gas sensing system using a plurality of gas sensor chips at respectively different temperatures.

### DESCRIPTION OF EMBODIMENTS

The embodiment will hereinafter be described in detail based on some drawings. In all the drawings for describing the embodiment, members (units) or functional blocks having the same function are indicated by the same reference numeral or another reference numeral relating thereto, and the repeated description thereof may be omitted. When there is a plurality of similar members (units) or functional blocks, a suffix is added to a reference numeral showing a generic name to indicate an individual or specific member or the like. In the following embodiment, a description on the same or similar portion is not repeated in principle unless otherwise necessary.

In the drawings used for the embodiment, even the cross-sectional view may be drawn without hatching to facilitate understanding of it. In addition, even a plan view may be hatched to facilitate understanding of it.

In the cross-sectional view and plan view, the size of each unit does not correspond to that of an actual device and a certain unit may be drawn with a relatively large size to facilitate understanding of the drawings. When a cross-sectional view and a plan view correspond to each other, a certain unit may be indicated with a relatively large size to facilitate understanding of the drawings.

### <Constitution of a Gas Sensing System According to First Embodiment>

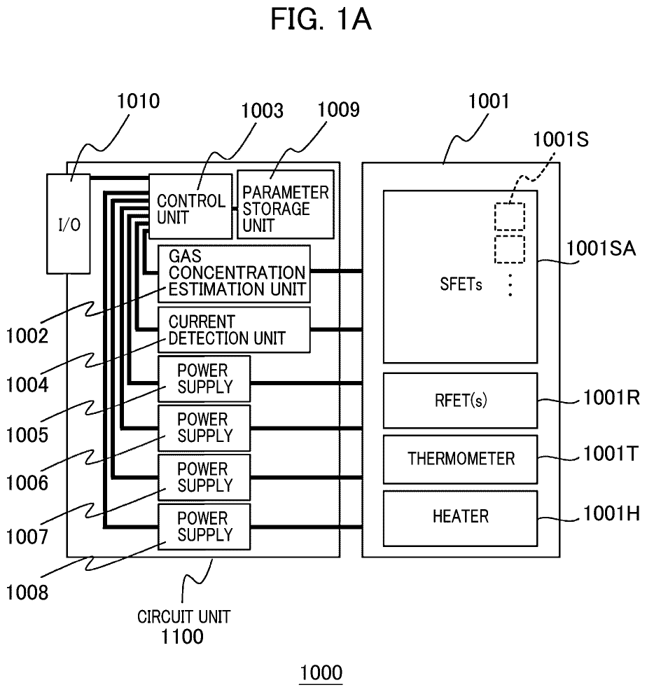

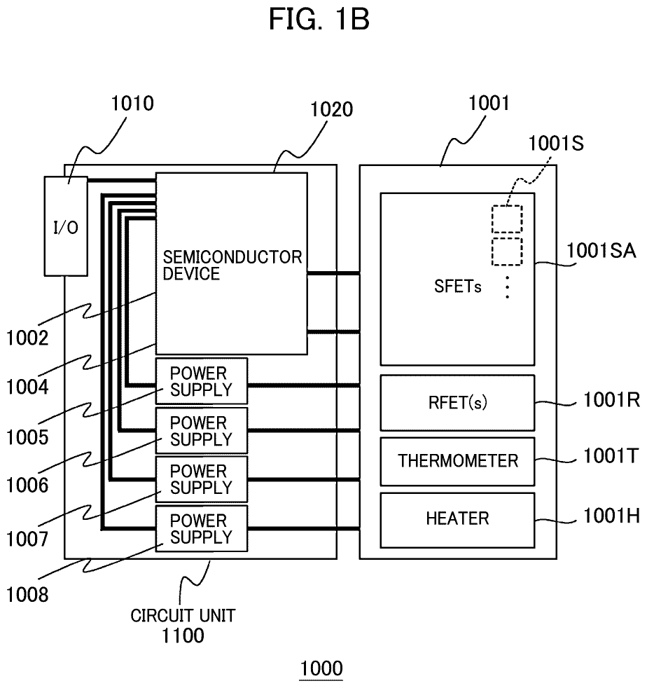

FIG. 1A shows one example of a gas sensing system of First Embodiment. As shown in FIG. 1A, a gas sensing system 1000 has a gas sensor chip 1001 and a circuit unit 1100.

The gas sensor chip 1001 has a sensor FET array 1001SA including a plurality of sensor FETs (which may hereinafter be called “SFETs”) 1001S, each an FET type sensor which is one of work function type sensors, and one or more reference FETs (which may hereinafter be called “RFETs”) 1001R. The gas sensor chip 1001 further has a thermometer unit (Thermometer) 1001T and a heater unit (Heater) 1001H. It is to be noted that “reference FET” is one example of “the first FET” in the present invention.

The sensor FET 1001S is one of work function type sensors (please refer to Non-Patent Literature 1 on details of the work function type sensor such as constitution and effect) equipped with a catalyst metal gate whose gate threshold voltage changes depending on the concentration of various gases contained in the atmosphere. The term “gate threshold voltage” means a gate (Gate) voltage at which a certain current flows between the drain (Drain) and the source (Source) of an FET while a predetermined voltage is applied between the drain and the source. In the present Example, a catalyst metal gate FET type gas sensor is used as this gas sensor element.

The reference FET 1001R is an FET element whose gate threshold voltage does not depend on the concentration of a gas in the atmosphere. The reference FET 1001R and the sensor FET array 1001SA are formed on the same gas sensor chip 1001. Comparison between the gate threshold voltage of the sensor FET 1001S and the gate threshold voltage of the reference FET 1001R makes it possible to carry out more accurate gas sensing taking, in consideration, the characteristic variation of FET caused by the temperature change of the gas sensor chip 1001. It is to be noted that the reference FET 1001R is not necessary when highly accurate gas sensing is required. On the contrary, when highly accurate gas sensing is required, a plurality of reference FETs 1001R may be formed for one gas sensor chip 1001 to refer to values of the plurality of reference FETs 1001R such as average or median of the gate threshold voltage.

The thermometer unit 1001T is a device, element, or member capable of measuring the temperature of the gas sensor chip 1001. In the present Example, the thermometer unit 1001T contains a diode element as one example. In the diode element, the relationship between an applied voltage and a current depends on the environmental temperature. The environmental temperature can be identified by making use of the aforesaid characteristic and detecting the current of the diode element.

The heater unit 1001H is a device, element, or member capable of heating the gas sensor chip 1001 and thereby elevating its temperature. In the present Example, the heater unit 1001H includes a heater wire (heating wire) as one example. The temperature of the gas sensor chip 1001 can be controlled by controlling the current to be applied to the heater wire and thereby adjusting the heat generation amount.

The circuit unit 1100 is electrically connected to the gas sensor chip 1001. The circuit unit 1100 has a gas concentration estimation unit 1002, a control unit 1003, a current detection unit 1004, power sources 1005 to 1008, a parameter storage unit 1009, and an I/O (Input/Output) unit 1010.

The gas concentration estimation unit 1002 estimates the gas concentration of plural types of gases based on the information obtained from the gas sensor chip 1001.

The current detection unit 1004 detects the current of various elements which the gas sensor chip 1001 has.

The power sources 1005 to 1008 apply a voltage to various elements or members (units) on the gas sensor chip 1001 and thereby supply them with electric power. The power sources 1005 to 1008 are supposed to be used properly depending on the place to which the power is supplied, which is however not necessary. The control unit 1003 is electrically connected to the power sources 1005 to 1008, the current detection unit 1004, the gas concentration estimation unit 1002, the parameter storage unit 1009, and the I/O unit 1010 and it controls each unit or communicates with each unit. The parameter storage unit 1009 stores various parameters necessary for the control or computation which the control unit 1003 carries out. The I/O unit inputs/outputs information and power between the control unit 1003 and the outside of the gas sensing system 1000.

The heater unit 1001H can heat the gas sensor chip 1001 to elevate its temperature by applying a voltage to the heater wire constituting the heater unit 1001H from the power sources 1005 to 1008 to cause a current flow. The control unit 1003 controls the power sources 1005 to 1008 to apply a certain voltage to the diode element constituting the thermometer unit 1001T. The current detection unit 1004 detects a current that flows in the diode element constituting the thermometer unit 1001T and outputs a detected current value to the control unit 1003 under conditions where the applied voltage to the diode element is constant. The parameter storage unit 1009 stores therein a parameter that defines the relationship between the current flowing in the diode element and the temperature when the current flows. The control unit 1003 refers to the parameter and determines the temperature of the gas sensor chip 1001 based on the detected current value. To maintain the temperature of the gas sensor chip 1001 at a target temperature, the control unit 1003 controls the power sources 1005 to 1008 to adjust the voltage to be applied to the heater unit 1001H.

The gas concentration estimation unit 1002 obtains the physical quantity relating to voltage-current characteristics of the sensor FET array 1001SA and the reference FET 1001R depending on the type or concentration of the gas and estimates the concentration of various gases based on the physical amount thus obtained. The treatment for estimating the concentration of various gases by the gas concentration estimation unit 1002 will be described in detail later. It is to be noted that the term “physical amount” is one example of “information” in the present invention.

FIG. 1B shows one example of the hardware constitution of the gas sensing system of First Embodiment. FIG. 1A indicates the control unit 1003, the gas concentration estimation unit 1002, the current detection unit 1004, and the parameter storage unit 1009 as functional blocks, respectively. These functional blocks are, for example, as shown in FIG. 1B, comprised of a semiconductor device 1020. The semiconductor device 1020 includes a processor and a memory such as CPU (Central Processing Unit), MPU (Micro Processor Unit), and MCU (Micro Controller Unit). The processor carries out a predetermined program stored in the memory, by which the semiconductor device 1020 functions as each of the aforesaid functional blocks.

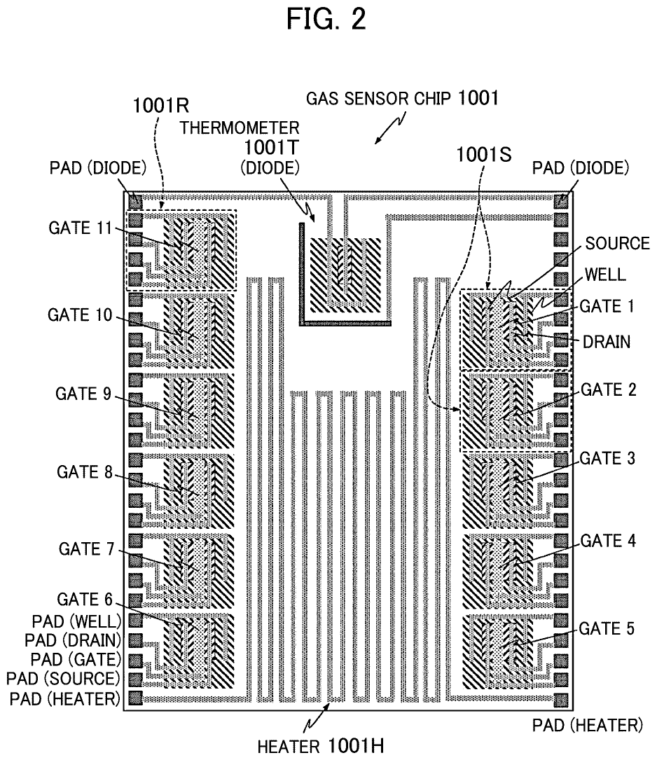

FIG. 2 is a plan view of the gas sensor chip of First Embodiment. As shown in FIG. 2, the gas sensor chip 1001 has, for example, ten sensor FETs 1001S (having gates indicated by Gates 1 to 10, respectively) and one reference FET 1001R (having a gate indicated by Gate 11). The sensor FETs 1001S and the reference FET 1001R each have a gate (Gate), a drain (Drain), a source (Source), and a well (Well) as a well-known FET has. The sensor FETs 1001S are each a catalyst metal gate FET type gas sensor element.

In the sensor FETs 1001S, the gate (Gate) has thereon a gas detection material comprised of a metal oxide layer and a catalyst metal layer. These sensor FETs 1001S are different from each other in any of the material, film thickness, structure, and crystal particle size of the metal oxide layer and the material, film thickness, structure, and crystal particle size of the catalyst metal layer. The gas detection material layers different in constitution differ in selectivity or sensitivity to a gas. Therefore, it is possible to measure the component pattern of a gas in the atmosphere containing plural types of gases by using a plurality of sensor FETs 1001S having thereon gas detection materials different in constitution and thereby measuring the gate threshold voltage of them.

The reference FET 1001R is an FET that does not show reactivity to the type or concentration of the gas in the atmosphere. The reference FET 1001R is, for example, constituted so that the gate that reacts with a gas is not exposed to the atmosphere. The reference FET 1001R basically has a constitution similar to that of the sensor FET 1001S except that the gate is not exposed to the atmosphere.

The gas sensor chip 1001 further has the heater unit 1001H and the thermometer unit 1001T. The heater unit 1001H is comprised of a heater wire and the heater wire is a wire comprised of a metal such as aluminum, tungsten, or platinum. The heater wire can elevate the temperature of the gas sensor chip 1001 to an environmental temperature or more by Joule heat generated by supplying power to it from the power sources 1005 to 1008 and thereby applying a current between both ends. For example, the heater wire constituting the heater unit 1001H can elevate the temperature of the gas sensor chip 1001 to a temperature higher by 100 °C or more from the environmental temperature. In addition, the temperature of the gas sensor chip 1001 can be estimated by measuring the resistance between both ends of the heater wire. The thermometer unit 1001T is comprised of a diode (Diode) element. As described above, the temperature of the gas sensor chip 1001 can be estimated from a current value that flows by the application of a certain voltage to the diode element. By keeping the gas sensor chip 1001 at a predetermined temperature, the electrical characteristics of the sensor FET and reference FET can be stably maintained as supposed.

The current detection unit 1004 shown in FIG. 1A measures, as described later, the current that flows in the sensor FET 1001S, the reference FET 1001R, the heater wire that constitutes the heater unit 1001H, and the diode element that constitutes the thermometer unit 1001T. The parameter storage unit 1009 shown in FIG. 1A stores therein, for example, voltage conditions to be applied to the sensor FET 1001S, the reference FET 1001R, the heater wire of the heater unit 1001H, and the diode element of the thermometer unit 1001T.

It is to be noted that in FIG. 2, each electrode of the element is connected to an electrode pad (Pad) corresponding thereto. The above constitution is one example to facilitate understanding of the constitution of the gas sensor chip 1001. Therefore, space saving may be achieved, for example, by making a plurality of electrode wires common and reducing the number of electrode pads.

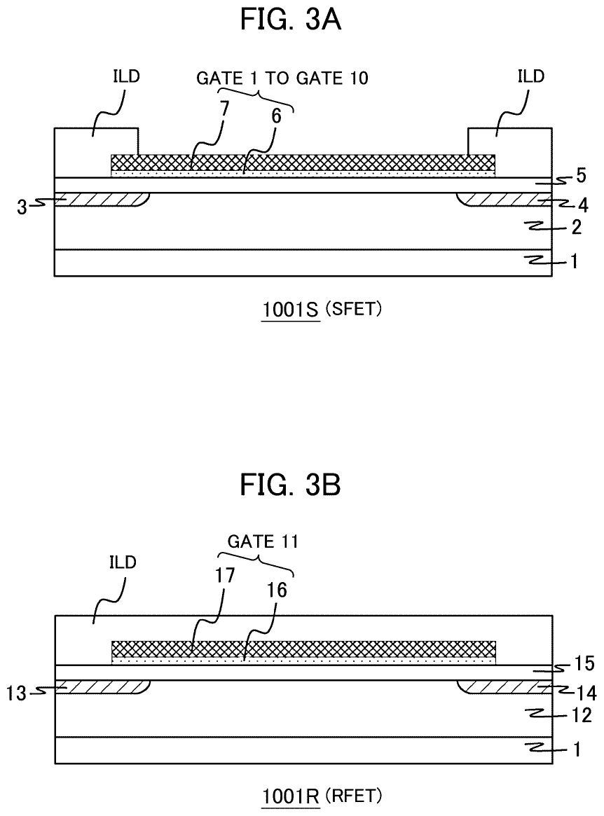

FIG. 3A schematically shows one example of the cross-section of a sensor FET (SFET). The major portion of the sensor FET 1001S is comprised of a semiconductor substrate 1, a well 2, a source layer 3, a drain layer 4, a gate insulating film layer 5, and a metal oxide layer 6 and a catalyst metal layer 7 which will be detection materials. The source layer and the drain layer are also called a “source diffusion layer” and a “drain diffusion layer”.

The metal oxide layer 6 and the catalyst metal layer 7 are covered with an interlayer insulating film ILD, but the interlayer insulating film ILD has an opening to expose the surface of the catalyst metal layer 7 to the atmosphere containing a detection target gas. For the semiconductor substrate 1, for example, silicon or silicon carbide (SiC) may be used. For the metal oxide layer 6, for example, titanium oxide, yttria-stabilized zirconia (YSZ), or the like may be used. As the catalyst metal layer 7, for example, a noble metal such as platinum, palladium, or iridium and in addition, nickel may be used. As shown in FIG. 2, the gas sensor chip 1001 has thereon a plurality of sensor FETs 1001S. Gas detection material layers of these sensor FETs 1001S are not the same and they are different from each other in constitution. Respective gas detection material layers in a plurality of sensor FETs 1001S are different, for example, in at least one of the type, film thickness, and crystal particle size of the material of the metal oxide layer 6 and the type, film thickness, and crystal particle size of the material of the catalyst metal layer 7.

FIG. 3B schematically shows one example of the cross-section of the reference FET (RFET). The major portion of the reference FET 1001R is comprised of a semiconductor substrate 1, a well 12, a source layer 13, a drain layer 14, a gate insulating film layer 15, and a metal oxide layer 16 and a catalyst metal layer 17 which will be detection materials. The catalyst metal layer 17 has a surface covered with the interlayer insulating film ILD and is therefore separated from the atmosphere containing a detection target gas. For the metal oxide layer 16 and the catalyst metal layer 17, the same material used for any of the sensor FETs 1001S may be used. To the wells 2 and 12, the source layers 3 and 13, the drain layers 4 and 14, the catalyst metal layers 7 and 17, the diode element constituting the thermometer unit 1001T, and the heater wire constituting the heater unit 1001H, a wiring layer comprised of a metal such as aluminum, tungsten, or platinum is connected to enable electricity supply from the power sources 1005 to 1008 shown in FIG. 1.

For each of the sensor FET 1001S and the reference FET 1001R, an N type FET is usable or a P type FET is usable. The gas sensor chip 1001 includes a plurality of sensor FETs 1001S and for each of them, an N type FET is usable or a P type FET is usable. Alternatively, some sensor FETs 1001S may be formed using an N type FET and the remaining sensor FETs 1001S may be formed using a P type FET. When one reference FET 1001R is mounted on the gas sensor chip 1001, it may be formed using an N type FET or a P type FET. When a plurality of reference FETs 1001R is formed in the gas sensor chip 1001, the combination pattern of the N type FET and the P type FET is the same as that of the sensor FET 1001S.

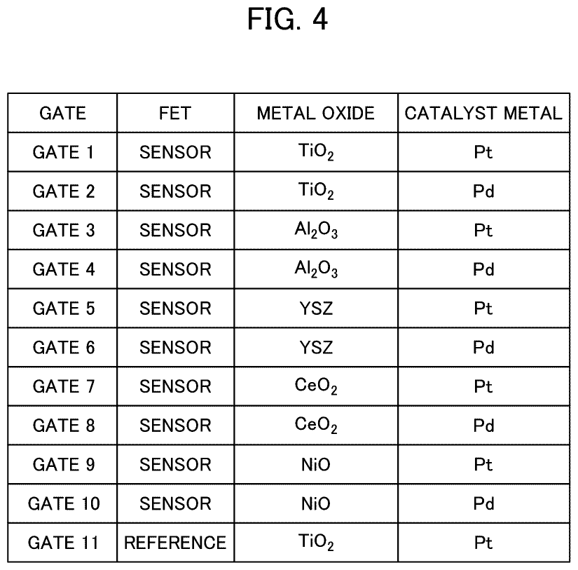

FIG. 4 shows one example of a material used for the gas detection material layer of the sensor FET and the reference FET. FIG. 4 shows examples in which the gas detection material layers of ten sensor FETs 1001S (having, as a gate, Gate 1 to Gate 10 in FIG. 2) use materials, which are different in type from each other, for the metal oxide layer 6 and the catalyst metal layer 7, respectively. Using such a combination allows the ten sensor FETs 1001S to have respectively different sensitivities to a detection target gas. Although not shown in the drawing, it is also possible to make sensitivities to a detection target gas different from each other, by forming the metal oxide layers 6 different in film thickness or crystal particle size from each other or forming the catalyst metal layers 7 different in film thickness or crystal particle size from each other. Since gate formation can be performed simultaneously by forming the gas detection material layer of the reference FET 1001R (having, as a gate, Gate 11 in FIG. 2) equal to that of any of the sensor FETs 1001S, making it possible to prevent an increase in the number of steps for producing a gas sensor chip.

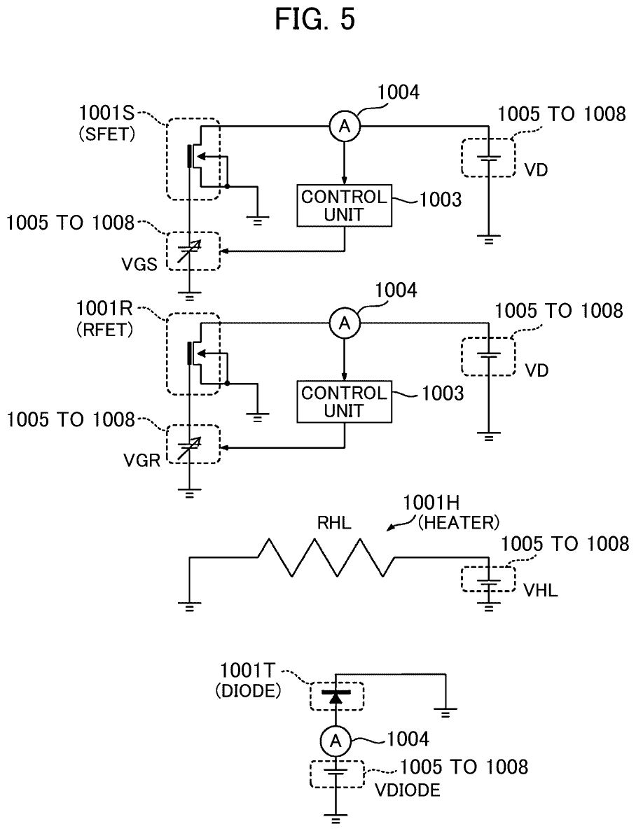

FIG. 5 shows one example of the operation during detection of the concentration of a detection target gas in a gas sensor chip using an FET type sensor. A current is applied to the heater wire constituting the heater unit 1001H (Heater) and by Joule heat generated at the resistor RHL of the heater wire, the gas sensor chip 1001 is heated to a predetermined temperature, for example, 100 °C. By elevating the temperature of the gas sensor chip from the ambient environmental temperature in such a manner and keeping it constant, the characteristics of the sensor can always be made stable. The power sources 1005 to 1008 apply a voltage of 0 V to the wells 2 and 12 and the source layers 3 and 13 of the sensor FET 1001S and the reference FET 1001R, while they apply a drain voltage VD to the drain layers 4 and 14. In addition, the power sources 1005 to 1008 apply a voltage VGR, which is a variable voltage, to the gate of the reference FET 1001R and applies a voltage VGS, which is a variable voltage, to the gate of the sensor FET 1001S. The current detection unit 1004 measures a current that flows in the drain terminal of each of the sensor FET 1001S and the reference FET 1001R. The control unit 1003 controls VGS and VGR so that they coincide with a certain current Ic. A difference between VGR and VGS at that time is designated as VGRS and is defined as in Equation 1.

VGRS=VGR−VGS (Equation 1)

A difference between VGRS(0) which is VGRS when the concentration of a detection target gas in the atmosphere is 0 and VGRS(X) which is VGRS when the concentration of a detection target gas is X is designated as ΔVg(X) and is defined as in Equation 2.

ΔVg(X)=VGRS(0)−VGRS(X) (Equation 2)

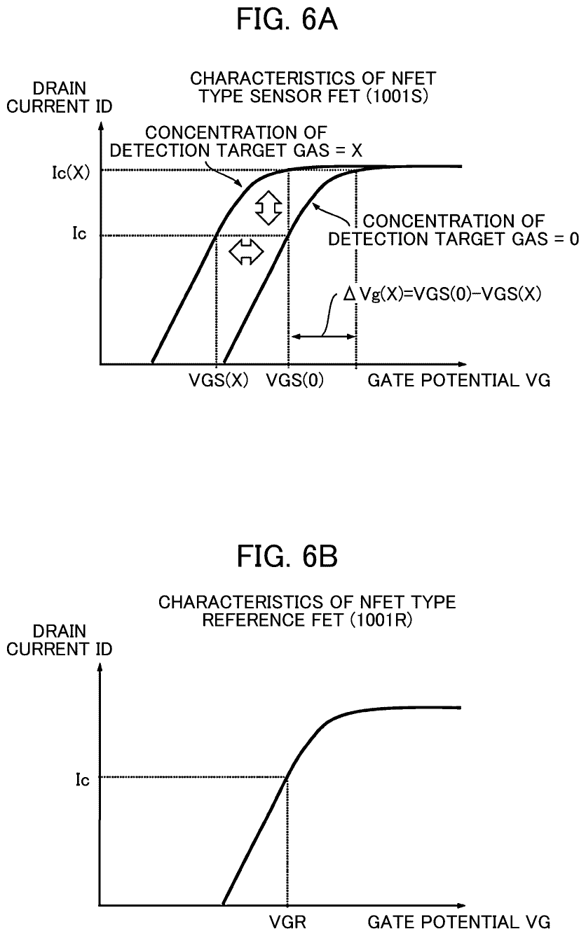

FIG. 6A shows gate voltage—drain current characteristics in an NFET type sensor FET. FIG. 6B shows gate voltage—drain current characteristics in an NFET type reference FET. It is to be noted that the term “gate voltage” is also called “gate potential”. In the NFET type sensor FET 1001S, since the catalyst metal layer 7 which is a gas detection material layer is exposed, the work function of the catalyst metal layer 7 changes with a detection target gas. The gate voltage—drain current characteristics are therefore different between when the concentration of a detection target gas is 0 and when it is X. Described specifically, as shown in FIG. 6A, with an increase in the concentration of a detection target gas, the characteristic curve showing the relationship between gate voltage and drain current shows a parallel shift in a voltage direction. As a result, the voltage VGS, which is a gate voltage when the current Ic, a threshold current, flows changes from VGS(0) which is VGS when the concentration of a detection target gas is 0 to VGS(X) which is VGS when the concentration of the detection target gas is X. On the other hand, in the reference FET 1001R, the catalyst metal layer 17 is covered with the interlayer insulating film ILD so that even a change in the gas concentration does not cause a change in the gate voltage—drain current characteristics. Described specifically, as shown in FIG. 6B, a current that flows by the application of a voltage VGR to the gate remains constant at Ic. As a result, the ΔVg(X) of Equation 2 corresponds to a change in gate threshold voltage of the sensor FET 1001S relative to the gas concentration X.

As shown in Equation 1, when VGRS which is a difference in gate voltage between the sensor FET 1001S and the reference FET 1001R is used, setting an appropriate value for the drain current ID that flows in the aforesaid drain terminal makes it possible to suppress the influence of the noise due to the variation of VGR and VGS caused by a temperature variation or the like.

It is to be noted that when the influence of such noise can be suppressed sufficiently or the influence of such noise is negligible and, for example, gas concentration detection with relatively high accuracy is not required, the reference FET 1001R is not always necessary. A difference between VGS(0) when the concentration of a detection target gas in the detection atmosphere is 0 and VGS(X) when the concentration of a detection targe gas is X is designated as ΔVg(X) and it is defined as in the following Equation 3.

ΔVg(X)=VGS(0)−VGS(X) (Equation 3)

The ΔVg(X) thus defined can be used as a gate threshold voltage shift (amount of change) of the sensor FET 1001S relative to the gas concentration X of the detection target gas. It is to be noted that FIG. 5 shows an example in which current measurement is performed at a drain terminal, but as will be described later, ΔVg may of course be obtained by measuring the current on the source terminal side.

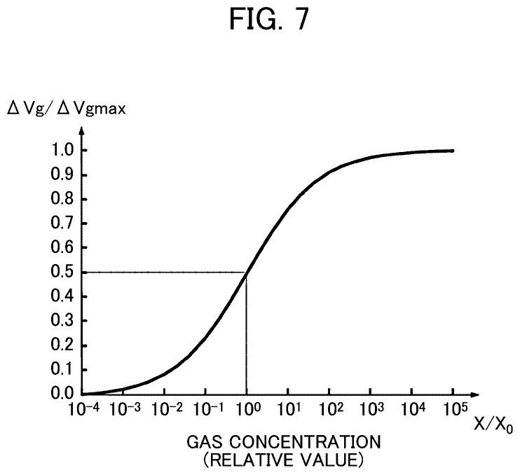

FIG. 7 shows one example of the relationship between a gas concentration and a gate threshold voltage shift in sensor FET. In FIG. 7, a saturated value of a gate threshold voltage shift ΔVgs(X) under the environment where the gas concentration X is sufficiently high is designated as ΔVgmax. FIG. 7 shows one example of the relationship between ΔVg and a gas concentration X when the FET type gas sensor is retained in the atmosphere of the gas concentration X for sufficiently long hours and an equilibrium state comes. The ΔVgmax may be a positive value or a negative value, depending on the type of a gas. The gas concentration X₀ when the ΔVgs(X) reaches 50% of the ΔVgmax changes by changing the type, film thickness or crystal particle size of the material of the metal oxide layer 6 or the type, film thickness, or crystal particle size of the material of the catalyst metal layer 7. Dependence of the gas concentration X₀ on the gate material changes by changing the type of a detection target gas. This means that the gate threshold voltage of the sensor FET changes depending on the type or concentration of the gas and it also changes even by a change in the type, film thickness, or crystal particle size of the material of at least one of the metal oxide layer 6 and the catalyst metal layer 7 constituting the gate material.

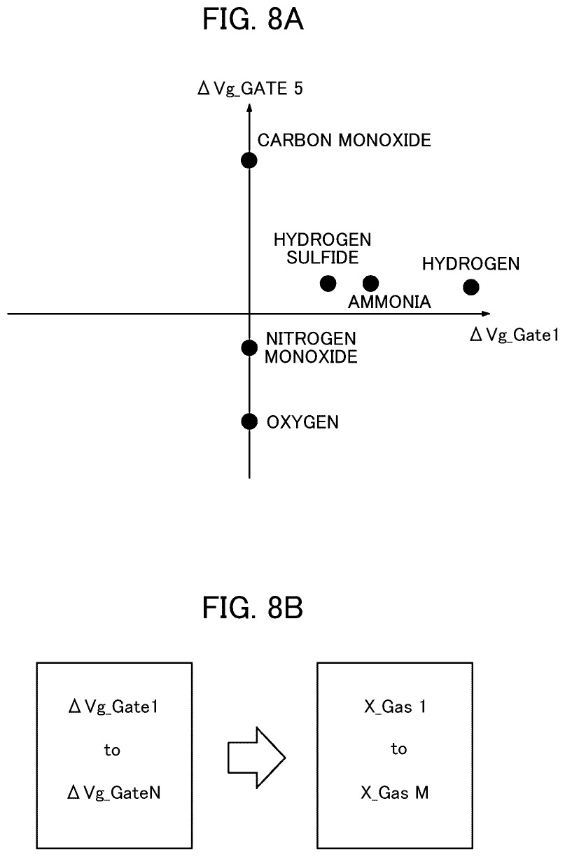

FIG. 8A shows one example of how two sensor FETs respond to some gases having a certain concentration. FIG. 8A uses, as an example of the type of the gas, hydrogen, hydrogen sulfide, ammonia, carbon monoxide, nitrogen monoxide, and oxygen FIG. 8A is obtained by plotting the data of various gases in a two-dimensional space comprised of ΔVg of two sensor FETs 1001S (used as examples are those having, as the gate, Gate 1 and Gate 5).

In general, the data of various gases are plotted in the space of the number of dimensions corresponding to the number of the sensor FETs 1001S and are distinguished. Described specifically, when N pieces of sensor FETs different from each other in the gas detection material of the gate are loaded on the gas sensor chip 1001, the data of various gases are potted in an N-dimensional space comprised of N pieces of ΔVgs and are distinguished. For example, in the example shown in FIGS. 2 to 4, since ten sensor FETs 1001S having a gate comprised of ten gas detection materials different from each other are loaded in the gas sensor chip 1001, respectively, the data of various gases are plotted in a ten-dimensional space comprised of ten ΔVgs and are distinguished. FIG. 8A shows a response to a single gas. The measurement results obtained using the atmosphere containing M types of gases are, at M≤N, plotted as a different point in the N-dimensional space comprised of N pieces of ΔVgs depending on the concentration of each of the gas types contained. At N<M, measurement results obtained using the atmosphere different in the type of gases and their concentrations may inevitably be plotted on the same point.

Based on the measurement results plotted in the aforesaid manner, in the case of a multi gas as well as a single gas, the atmospheres different in the type of a gas and the gas concentration can be distinguished as different regions of the N-dimensional space. Described specifically, it becomes possible to determine the type of gases and their concentrations in the atmosphere by preliminarily studying the pattern of an amount of change, in each gas and at each concentration, that affects the gate threshold voltage of each of respectively different N pieces of sensor FETs and solving simultaneous equations obtained from the aforesaid measurement results. Alternatively, it becomes possible to determine the type of gases and their concentrations in the atmosphere from the aforesaid measurement results by using the artificial intelligence (AI) which has learned the pattern of a shift amount of each gate threshold voltage of respectively different N pieces of sensor FETs and the correspondence between the type of gases and their concentrations in the atmosphere.

FIG. 8B is a view for describing that the concentration of M types of gases can be estimated using respectively different N pieces of sensor FETs. Suppose that the component pattern of the atmosphere containing M types of gases is measured using a plurality of sensor FETs 1001S having thereon respectively different N types of gas detection material layers. As shown in FIG. 8B, at M≤N, it is also possible to estimate M types of gas concentrations X_Gas1 to X_GasM based on the found values ΔVg_Gate1 to ΔVg_GateN of the threshold voltage shift of N pieces of sensor FETs obtained from the N pieces of sensor FETs. The ΔVg_Gate1 to ΔVg_GateN are each a function of M types of gas concentrations X_Gas1 to X_GasM. Strictly speaking, at M≤N, since the number of equations (N pieces of ΔVg_Gate1 to ΔVg_GateN) is larger than the number of variables (M pieces of X_Gas1 to X_GasM), the value of the variables (X_Gas1 to X_GasM) is sometimes not determined. In such a case, it is possible to apply a method of estimating the X_Gas1 to X_GasM so as to minimize the error between the found value of ΔVg_Gate1 to ΔVg_GateN and the calculated value ΔVg_Gate1_Calc to ΔVg_GateN_Calc of a threshold voltage shift determined from the gas concentrations X_Gas1 to X_GasM. The error can be calculated, for example, by using the sum of squares of a difference between the found value and calculated value of N pieces of the threshold voltage shift, in short, by using the following Equation 4.

$$Δ Vg\_Sum = \qquad ( Equation 4 )$$

$${( Δ Vg\_Gate1 - Δ Vg\_Gate1 \_Calc )}^{2} +$$

$${( Δ Vg\_Gate2 - Δ Vg\_Gate2 \_Calc )}^{2} +$$

$${( Δ Vg\_Gate3 - Δ Vg\_Gate3 \_Calc )}^{2} +$$

$$…$$

$${( Δ Vg\_GateN - Δ Vg\_GateN \_Calc )}^{2}$$

The X_Gas1 to X_GasM is estimated to minimize the ΔVg_Sum indicated by Equation 4. The gas concentration estimation unit 1002 estimates the concentration of various gases by carrying out treatment based on the aforesaid principle based on the information available from the gas sensor chip 1001.

### Effect by First Embodiment

The gas sensor chip according to First Embodiment has a semiconductor substrate and a plurality of gas detection material layers which are formed on the semiconductor substrate. The plurality of gas detection material layers has a metal oxide layer and a catalyst metal layer formed over the metal oxide layer and it is configured to expose the catalyst metal layer to the atmosphere. In the above gas sensor chip, the gas detection material layers are different from each other in the combination of the type, film thickness, and polycrystal particle size of the material of the metal oxide layer and the type, film thickness, and polycrystal particle size of the material of the catalyst metal layer. This means that the gas detection material layers are different from each other in at least one of the type, film thickness, and polycrystal particle size of the material constituting the gas detection material layer.

The gate threshold voltage of the gas sensor itself changes depending on the concentration of a detection target gas. Therefore, work function type sensors included in the sensor array can have different selectivities to plural gas types contained in the atmosphere, respectively. Therefore, by using one gas sensor chip, the concentration of many types of gases can be detected or the type of smell can be determined based on the gas component pattern detected.

According to such a gas sensor chip of First Embodiment, a semiconductor substrate has thereon plural types of work function type sensors which are different from each other in the characteristics for the type of a gas or concentration of the gas and have a simple structure similar to that of MOSFET. Therefore, a chip capable of sensing plural types of gases can be produced at a reduced cost. In other words, it is not necessary to prepare plural types of gas sensors individually at a high cost or to manufacture a chip having a costly structure. As a result, sensing of a gas in the atmosphere can be achieved at a low cost. In addition, the work function type sensor has a simple structure so that stable operation can be expected from it and therefore it has high reliability.

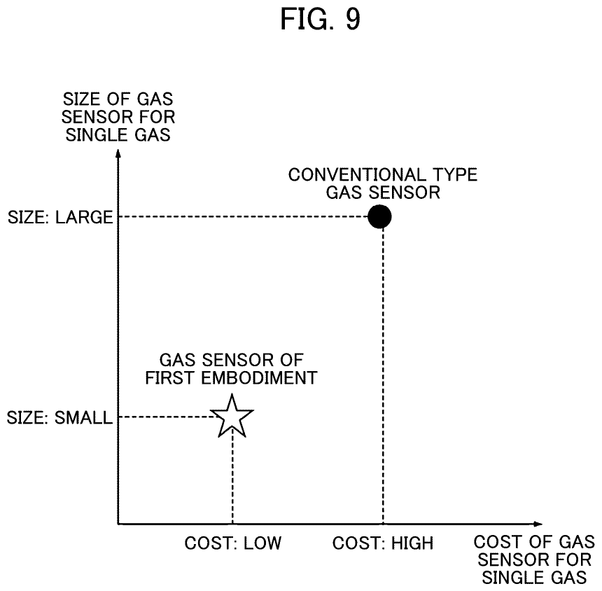

FIG. 9 shows an example of comparison results between the gas sensor of First Embodiment and a conventional gas sensor in the size and cost of the gas sensor per type of gas. By using the technology of First Embodiment, it is possible to integrate a plurality of sensor FETs having respectively different gas detection materials on one chip. The size of a gas sensor per type of a detectable gas can be made smaller in First Embodiment than in a conventional technology and a cost per type of a detectable gas can be reduced.

The gas sensing system according to First Embodiment has a gas sensor chip, a heater unit that heats the gas sensor chip to elevate its temperature, and a control unit that controls the gas sensor chip and the heater unit. The gas sensor chip has a semiconductor substrate and a plurality of gas detection material layers formed on the semiconductor substrate at positions different from each other. The gas detection material layers each has a metal oxide layer and a catalyst metal layer formed over the metal oxide layer. They are each constituted to expose the catalyst metal layer to the atmosphere. The gas detection material layers are different from each other in the combination of the type, film thickness, and crystal particle size of the metal oxide layer and the type, film thickness, and crystal particle size of the catalyst metal layer. The control unit controls the heater unit to adjust the gas sensor chip to have a predetermined temperature and detects the gas in the atmosphere based on the information available using the gas detection material layers.

Such a gas sensing system can provide an effect similar to that of the aforesaid gas sensor chip and for example, gas sensing in the atmosphere can be achieved at a low cost.

### Modification Example of First Embodiment

In examples shown in FIGS. 2 to 8, a gas sensor chip has thereon electrode pads for respectively supplying electricity to a source terminal, a drain terminal, a well terminal and a gate terminal of the sensor FETs and reference FET. Apart from the aforesaid examples, the terminals may be shared by the sensor FETs themselves, the reference FETs themselves, or the sensor FET and the reference FET. For example, a well terminal may be shared by P type PETs (which may be called PFETs) themselves or N type FETs (which may be called NFETs) themselves. When operation is carried out while fixing the source potential at 0 V, the source terminal may be shared. By sharing the terminal of FETs mounted on a gas sensor chip or sharing the wire to be connected to the terminal, it is possible to reduce the number of electrode pads, reduce the chip area of the gas sensor, and reduce the cost per the number of sensor FETs. It is to be noted that “electrode pad” which is connected to a source terminal or a drain terminal and to which electricity is supplied from a power source is one example of “power supply terminal” in the present invention.

As an element to be loaded on a gas sensor chip, in addition to the sensor FET and the reference FET, a selection transistor (Selection Transistor) for selecting, from these FETs, a FET which will be a measurement target of a gate threshold voltage may be included. Using the selection transistor makes it possible to select the terminals of a plurality of sensor FETs with a small number of electrode terminals and thereby operate them. By controlling the switching ON/OFF of the selection transistor, it is possible to switch the sensor FET thus selected at high speed, collect the information of the gate threshold voltage shift of each sensor FET, and measure the concentration of a plurality of gas types in the atmosphere.

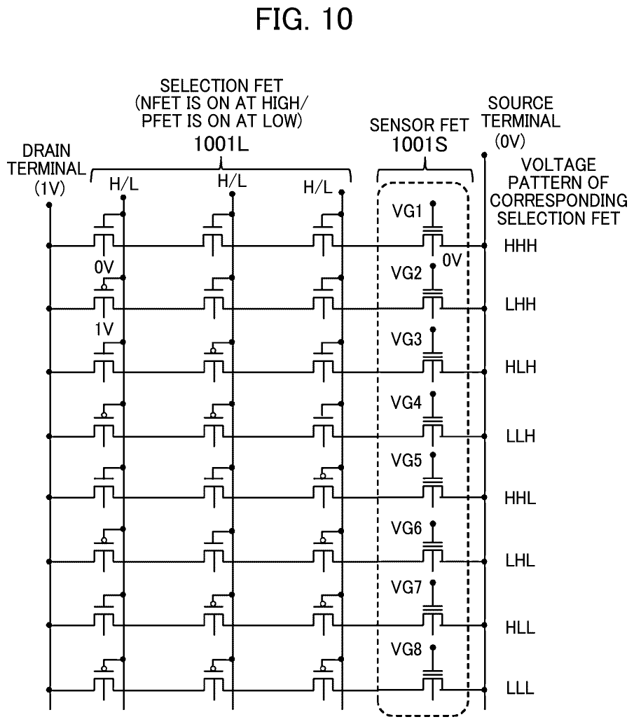

FIG. 10 shows the first example of an FET type gas sensor array using a selection transistor. In the example shown in FIG. 10, the sensor FETs 1001S are all comprised of an NFET, the sensor FETs 1001S share a well terminal and a source terminal, and the sensor FETs 1001S independently have a gate terminal and share a drain terminal via a selection FET 1001L. Here, a selection FET (Selection FET) is used as the selection transistor. It is to be noted that “Selection FET” is one example of the “second FET” in the present invention.

The well terminal, the source terminal, the drain terminal, and the gate terminal are connected to the circuit unit 1100 shown in FIG. 1. To measure the gate threshold voltage of each sensor FET, it is necessary to measure the drain current or source current of each sensor FET. When the sensor FETs share the drain terminal without the selection FET in the circuit shown in FIG. 10, they are placed in parallel between the drain terminal and the source current. This makes impossible to distinguish which sensor FET a drain current (or a source current) to be measured has flowed in. In short, it becomes impossible to measure the gate threshold voltage. The selection FET is therefore placed to enable connection of the drain terminal to only one of the plurality of sensor FETs.

In the circuit shown in FIG. 10, twenty-four selection FETs 1001L are used for eight sensor FETs 1001S. In addition, each sensor FET 1001S is connected to a drain terminal via a selection FET series circuit having three selection FETs connected in series. As shown in FIG. 10, three selection FETs to be connected to the top stage sensor FET 1001S are all comprised of an NFET, while three selection FETs to be connected to the bottom stage sensor FET 1001S are all comprised of a PFET. With respect to the sensor FETs 1001S on the other stages, three selection FETs 1001L to be connected to each other include both an NFET and a PFET. The gates of the eight selection FETs 1001L in the first column from the left are connected to each other, the gates of the eight selection FETs 1001L in the second column from the left are connected to each other, and the gates of the eight selection FETs 1001L in the third column from the left are also connected to each other.

To the gate of the selection FETs 1001L in each row, a voltage corresponding to either one of two values, that is, High or Low, is applied. The NFET is turned ON when the gate is High (H) and is turned OFF when the gate is Low (L). On the contrary, the PFET is turned OFF when the gate is High (H) and is turned ON when the gate is Low (L). By placing as shown in FIG. 10, when all the gates of the selection FETs in three columns are set High (H), only the sensor FET on the top stage is connected to the drain terminal. There are eight patterns of High and Low to be applied to three gates of the selection FET series circuit and they are HHH, LHH, HLH, LLH, HHL, LHL, HLL, and LLL. According to these eight patterns, any one of the eight sensor FETs is connected to the drain terminal.

As shown in FIG. 10, when the number of selection FETs connected in series is three, eight (23) sensor FETs can share a drain terminal. The number of sensor FETs capable of sharing a drain terminal increases by increasing the number of selection FETs to be connected in series and in general, 2^{N} pieces of sensor FETs can share a drain terminal by connecting N pieces of selection FETs in series. When N pieces of selection FETs are used, the number of electrode pads increases by N, which is the number of the gate terminals of the selection FET, but 2^{N} pieces of sensor FETs can share a drain terminal and the number of the electrode pad can therefore be reduced to one. It is therefore possible to decrease the necessary number of electrode pads by (2^{N}−1−N) when selection FETs are used compared with when the selection FET are not used. At 3≤N, the number of electrode pads can be made smaller when selection FETs are used and with an increase in the number of N, an effect of decreasing the number of electrode pads is marked.

It is to be noted that in the example of FIG. 10, the sensor FETs 1001S are comprised of an NFET. Even when the sensor FETs 1001S are comprised of a PFET, N pieces of selection FETs connected in series may be used similarly. The effect obtained in the above case is also similar to the case where the sensor FET 1001S is comprised of an NFET.

In the example of FIG. 10, the selection FET 1001L is comprised of both an NFET and a PFET, but the constitution is not limited thereto.

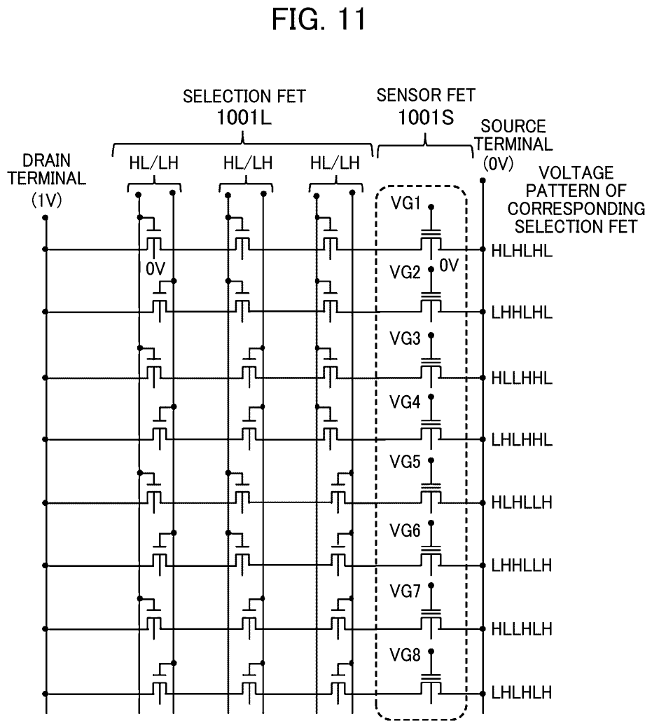

FIG. 11 shows the second example of an FET type gas sensor array using a selection transistor. As shown in FIG. 11, all the selection FETs may be comprised of an NFET having the conductivity type same as that of the sensor FET. In the example of FIG. 11, the number of gates shared by all the selection FETs is six and two gates form a pair. A High voltage is applied to one of the two gates of the pair and a Low voltage is applied to the other gate. There are eight voltage patterns to be applied to the six gates and the H/L patterns described in order from the gate on the left side are HLHLHL, LHHLHL, HLLHHL, LHLHHL, HLHLLH, LHHLLH, HLLHLH, and LHLHLH. Depending on each of the H/L patterns of the voltage applied to the six gates, one of eight sensor FETs 1001S corresponding to the pattern is connected to a drain terminal.

As shown in FIG. 11, when the number of selection FETs connected in series is three, eight (23) sensor FETs 1001S may share a drain terminal. With an increase in the number of selection FETs connected in series, the number of sensor FETs 1001S capable of sharing a drain terminal may increase. In general, by connecting N pieces of selection FETs in series, 2^{N} pieces of sensor FETs 1001S may share a drain terminal. By using N pieces of selection FETs 1001L connected in series, the number of electrode pads increases by the number corresponding to 2^{N} of the gate terminals of selection FETs 1001L. Since 2^{N} pieces of sensor FETs 1001S can share a drain terminal and the number thereof can be reduced to one, the necessary number of electrode pads decreases by (2^{N}−1−2N) compared with when selection FETs 1001L are not used. At 3≤N, the number of electrode pads can be made smaller when selection FETs 1001L are used and with an increase in the number N, an effect of decreasing the number of electrode pads is marked. Compared with the constitution of the circuit shown in FIG. 10, the constitution of the circuit shown in FIG. 11 has a smaller effect for reducing the number of electrode pads, in other words, a smaller effect for reducing the area of the gas sensor chip. In the constitution of the circuit shown in FIG. 11, however, the circuit of the selection FET 1001L can be comprised only of an NFT so that this constitution has a merit of simplifying the layout of the circuit and decreasing the number of steps in the production of a gas sensor chip.

In the example shown in FIG. 11, the sensor FET 1001S and the selection FET 1001L are each comprised of an NFET. Also when the sensor FET 1001S and the selection FET 1001L are each comprised of a PFET, N pieces of selection FETs may be connected in series. The effect is also similar to that obtained by constituting the sensor FET 1001S and the selection FET 1001L from an NFET.

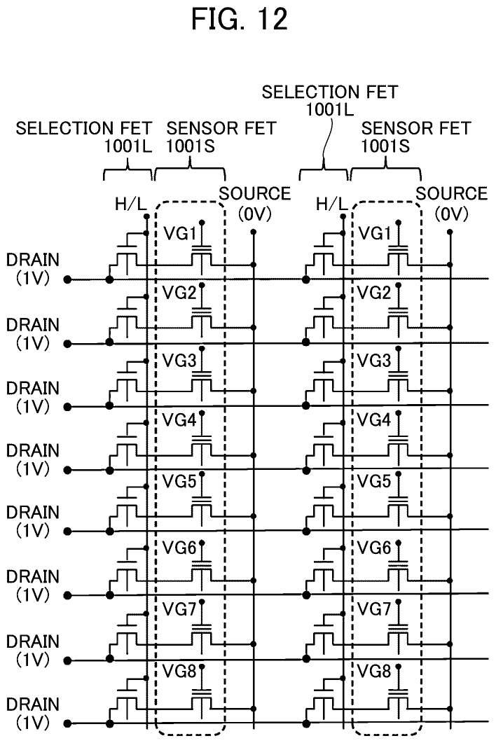

FIG. 12 shows the third example of an FET type gas sensor array using a selection transistor. In the example of FIG. 10 and the example of FIG. 11, plural sensor FETs 1001S are arranged in a one-dimensional direction, but as shown in FIG. 12, the sensor FETs 1001S may be arranged two-dimensionally. The example of FIG. 12 shows eight independent drain terminals, two gates shared by a plurality of selection FETs 1001L, and sixteen arrays comprised of sensor FETs 1001S. The sensor FETs 1001S and the selection FETs 1001L are both comprised of an NFET. Since there are eight independent drain terminals, eight sensor FETs 1001S may be selected simultaneously. By setting one of two gates shared by the selection FETs 1001L High and setting the other one Low, it is possible to select eight sensor FETs 1001S and measure the gate threshold voltage.

In the example of FIG. 12, an example of an array having 8×2 sensor FETs 1001S is shown. The number of independent drain terminals which can be arranged is more than eight and the number of gates shared by selection FETs 1001L can be arranged while increasing the number to more than 2. Supposing that the number of independent drain terminals is Nd and the number of gates shared by selection FETs is Ns, the number of sensor FETs 1001S is Nd×Ns. When selection FETs 1001L are not used, the necessary number of drain terminals is Nd×Ns, but the number of drain terminals can be reduced to Nd by using Ns pieces of gates shared by selection FETs 1001L. In addition, by using selection FETs 1001L, the number of electrode pads can be reduced by Nd×Ns−Nd−Ns. For example, at Ns=2, the number of electrode pads can be reduced when 3 Nd. For example, at 3≤Ns, the number of electrode pads can be reduced when 2≤Nd.

In the constitution in the example of FIG. 10 and the example of FIG. 11, when a current flows between a source terminal and a drain terminal, a current flows in N pieces of selection FETs 1001L connected in series. Then, in measuring the drain current of sensor FETs 1001S, the resistance of N pieces (3≤N) of selection FETs 1001L connected in series has an influence as parasitic resistance. In the constitution in the example of FIG. 12, on the other hand, since only one selection FET 1001L becomes parasitic resistance in measuring a drain current of sensor FETs 1001S, the influence of parasitic resistance of selection FETs 1001L can be made smaller than that in the constitution in the examples of FIG. 10 and FIG. 11.

In the example of FIG. 12, sensor FETs 1001S and selection FETs 1001L are comprised of an NFET. Even when sensor FETs 1001S and selection FETs 1001L are comprised of a PFET, an array of sensor FETs 1001S may be formed. Effects of the above constitution is similar to those obtained by constituting sensor FETs 1001S and selection FETs 1001L from an NFET.

In the circuit example described referring to FIG. 10 to FIG. 12, an FET is used as a selection transistor, but alternatively, another transistor such as a bipolar type transistor may be used.

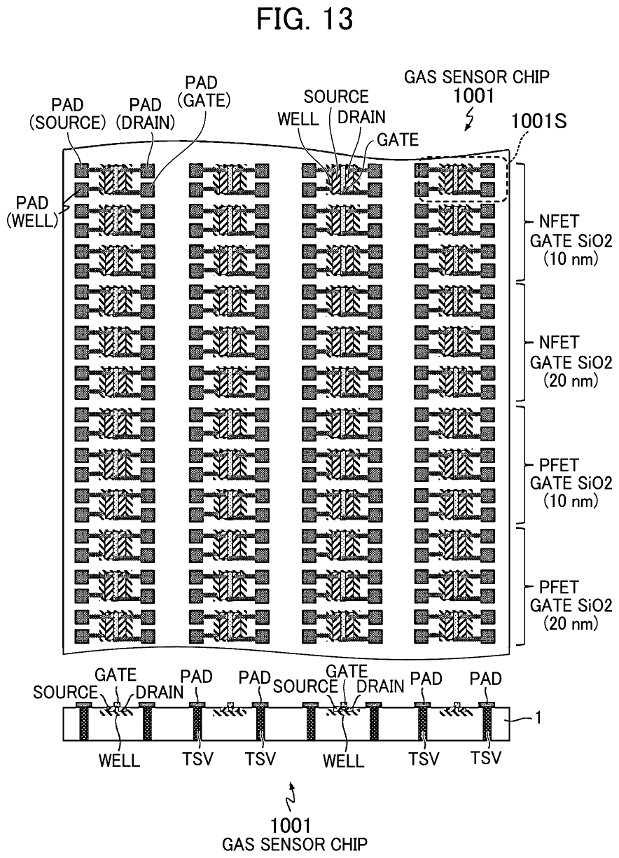

FIG. 13 shows one example of an FET type gas sensor array using TSV. FIG. 13 shows, on the upper side, a front view of the major portion of a gas sensor chip 1001 having the gas sensor array of the present example and, on the bottom side, a cross-sectional view of the gas sensor chip 1001. In the examples of FIG. 10 to FIG. 12, the array of sensor FETs 1001S is constituted using selection FETs 1001L, but as shown in FIG. 13, it is possible to supply power to the source terminal, drain terminal, well terminal, and gate terminal of a plurality of sensor FETs 1001S while using a through electrode (Through-Silicon Via: TSV) in a semiconductor substrate 1. According to the constitution as shown in FIG. 13, it is possible to place many sensor FETs 1001S without forming a selection FET on the surface of the gas sensor chip 1001 on which the sensor FETs 1001S are formed and without being affected by the limitation in wiring layout.

In the example of FIG. 13, the gas sensor chip 1001 has thereon sensor FETs 1001S comprised of an NFET and sensor FETs 1001S comprised of a PFET. Further, the sensor FETs 1001S comprised of an NFET use two thicknesses as a thickness of a silicon oxide film (SiO₂ film) serving as a gate insulating film. Similarly, the sensor FETs 1001S comprised of a PFET use two thicknesses as a thickness of a silicon oxide film (SiO₂ film) serving as a gate insulating film. In the example of FIG. 13, these two thicknesses are 10 nm and 20 nm, respectively. It is to be noted that as the thickness of the gate insulating film, three or more thicknesses may of course be used. It is to be noted that the “silicon oxide film” is one example of “an impurity layer” in the present invention. The term “SiO₂” may be indicated by SiO2” to improve the visibility in the drawings.

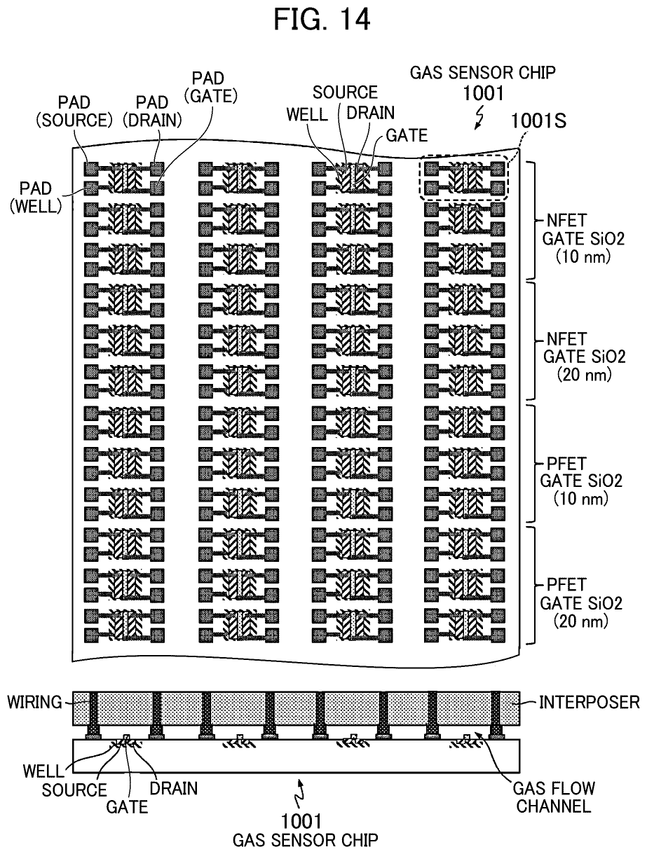

FIG. 14 shows one example of an FET type gas sensor array using an interposer. FIG. 14 shows, on the upper side, a front view of the major portion of a gas sensor chip 1001 having the gas sensor array of the present example and, on the bottom side, a cross-sectional view of the gas sensor chip 1001. As shown in FIG. 14, power can be supplied to the source terminal, the drain terminal, the well terminal, and the gate terminal of a plurality of sensor FETs 1001Ss by using an interposer (Interposer). As in the example shown in FIG. 13, it is possible to place many sensor FETs 1001S without forming a selection FET on the surface of the gas sensor chip 1001 on which the sensor FETs 1001S are formed and without being influenced by the limitation in wiring layout.

In the example of FIG. 14, similar to the example of FIG. 13, the gas sensor chip 1001 has thereon sensor FETs 1001S comprised of an NFET and sensor FETs 1001S comprised of a PFET. Further, the sensor FETs 1001S comprised of an NFET use two thicknesses as a thickness of a silicon oxide film (SiO₂ film) serving as a gate insulating film. Similarly, the sensor FETs 1001S comprised of a PFET use two thicknesses as a thickness of a silicon oxide film (SiO₂ film) serving as a gate insulating film. As the thickness of the gate insulating film, three or more thicknesses may of course be used.

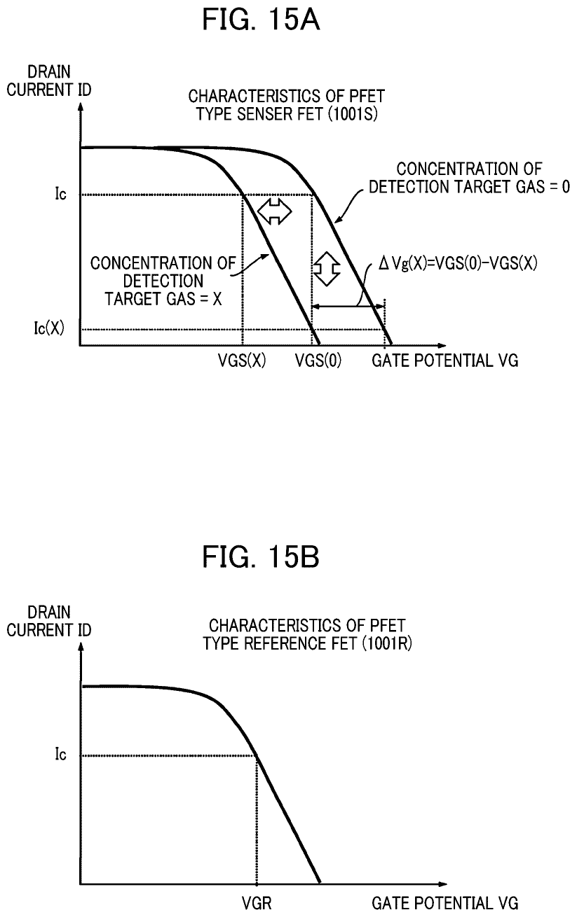

The gate voltage—drain current characteristics of a PFET type sensor FET will hereinafter be described. FIG. 15A shows gate voltage—drain current characteristics of a PFET type sensor FET. FIG. 15B shows gate voltage—drain current characteristics of a PFET type reference FET. The gate voltage—drain current characteristics of an NFET type sensor FET are as described in FIG. 6.

In the PFET type sensor FET 1001S, similar to the NFET type, the catalyst metal layer 7, which is a gas detection material layer, is exposed so that the work function of the catalyst metal layer 7 changes depending on a detection target gas and gate voltage—drain current characteristics differ between when the concentration of the detection target gas is 0 and when it is X. Described specifically, as shown in FIG. 15A, when the concentration of the detection target gas is high, the characteristic curve showing the relationship between gate voltage and drain current shows a parallel shift in a voltage direction. As a result, the voltage VGS, which is a gate voltage when a current Ic, a threshold current, flows changes from VGS(0) which is VGS when the concentration of the detection target gas is 0 to VGS(X) which is VGS when the concentration of the detection target gas is X. On the other hand, in the reference FET 1001R, the catalyst metal layer 17 is covered with the interlayer insulating film ILD so that even a change in the gas concentration does not cause a change in the gate voltage—drain current characteristics. Described specifically, as shown in FIG. 15B, a current that flows by the application of a voltage VGR to the gate remains constant at Ic. As a result, the ΔVg(X) of Equation 2 corresponds to a gate threshold voltage shift (amount of change) of the sensor FET 1001S relative to the gas concentration X.

Even if the sensor FET is comprised of a PFET instead of an NFET, the drain current—gate voltage characteristic curve similarly shows a parallel shift in a voltage direction. But when a current is measured while keeping the gate voltage constant, increase or decrease of the drain current in response to the same gas is reversed between the NFET and the PFET. In the NFET, when a gate threshold voltage shifts to the negative side and the gate voltage is constant, an absolute value of the drain current increases and when a gate threshold voltage shifts to the positive side and the gate voltage is constant, an absolute value of the drain current decreases. On the other hand, in the PFET, when a gate threshold voltage shifts to the negative side and the gate voltage is constant, an absolute value of the drain current decreases and when a gate threshold voltage shifts to the positive side and the gate voltage is constant, an absolute value of the drain current increases.

By making use of the aforesaid property and using the NFET type sensor FET and the PFET type sensor FET in combination, it is possible to constitute a device that determines, with respect to a certain gas type, whether or not the gas concentration in the atmosphere is in a predetermined concentration range, for example, 1 ppm to 10 ppm. Here, a gas type whose gate threshold voltage shifts in a negative voltage direction depending on the gas concentration will be considered as one example.

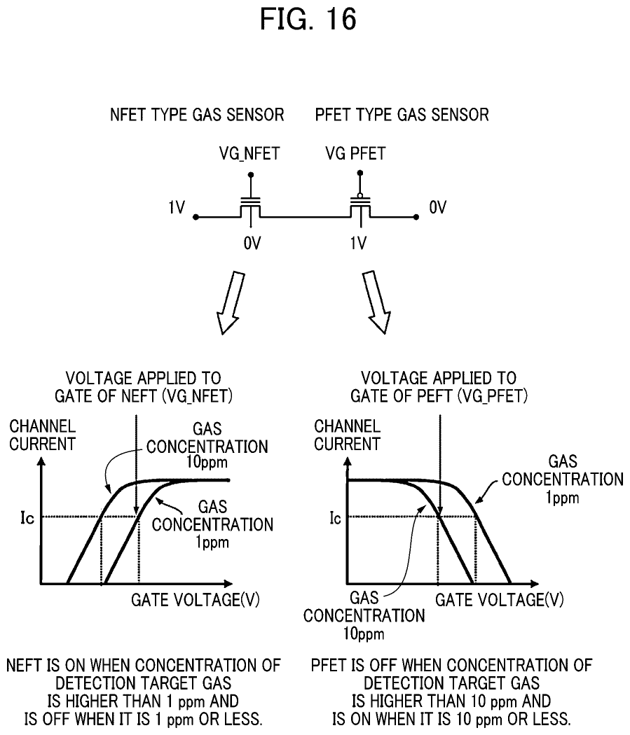

FIG. 16 shows the first example of a device that determines a gas concentration by using an NFET type sensor FET and a PFET type sensor FET in combination. For example, as shown in FIG. 16, in a device in which the NFET type sensor FET and the PFET type sensor FET are connected in series, 1V and 0V are applied to the terminals on both ends of the device. One V is applied to the well of the PFET to prevent application of a forward bias between a source drain layer and the well and 0 V is applied to the well of the NFET. A voltage VG_NFET is applied to the gate of the NFET to allow a current Ic, which is a determination current, to flow at a gas concentration of 1 ppm. A voltage VG_PFET is applied to the gate of the PFET to allow a current Ic, which is a determination current, to flow at a gas concentration of 10 ppm.

In such a device, when the gas concentration is lower than 1 ppm, a change in the gas concentration makes the current of the NFET smaller than Ic so that the device as a whole is in OFF state, though the PFET is in ON state. When the gas concentration is between 1 ppm and 10 ppm, the current of each of the NFET and the PFET becomes larger than Ic and the device as a whole is in ON state. When the gas concentration is higher than 10 ppm, the current of the PFET becomes smaller than Ic and the device as a whole is in OFF state, though the NFET is in ON state. Due to such operations of the NFET and PFET, only when the gas concentration is between 1 ppm and 10 ppm, the device as a whole is in ON state and therefore, a device that allows a current to flow between both ends can be obtained.

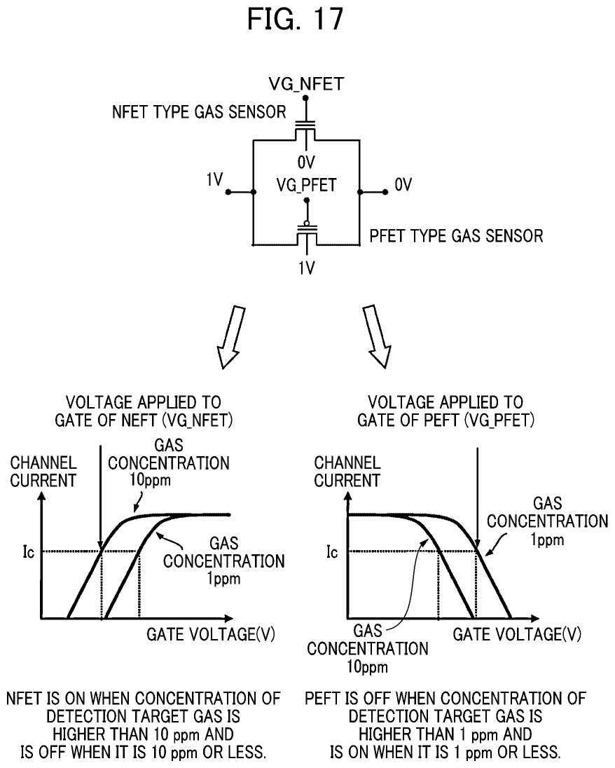

FIG. 17 shows the second example of the device that determines a gas concentration by using an NFET type sensor FET and a PFET type sensor FET in combination. For example, as shown in FIG. 17, in a device in which the NFET type sensor FET and the PFET type sensor FET are connected in parallel to each other, 1V and 0V are applied to the terminals on both ends of the device. One V is applied to the well of the PFET to prevent application of a forward bias between a source drain layer and the well and 0 V is applied to the well of the NFET. A voltage VG_NFET is applied to the gate of the NFET to allow a current Ic, which is a determination current, to flow at a gas concentration of 10 ppm. A voltage VG_PFET is applied to the gate of the PFET to allow a current Ic, which is a determination current, to flow at a gas concentration of 1 ppm.

In such a device, when the gas concentration is lower than 1 ppm, a change in gas concentration increases the current of PFET to more than Ic so that the device as a whole is in ON state, though the NFET is in OFF state. When the gas concentration is between 1 ppm and 10 ppm, the current of each of the NFET and the PFET becomes smaller than Ic and the device as a whole is in OFF state. When the gas concentration is larger than 10 ppm, the current of the NFET becomes larger than Ic and the device as a whole is in ON state, though the PFET is in OFF state. By such operations of the NFET and PFET, it is possible to manufacture a device which is as a whole in OFF state only when the gas concentration is between 1 ppm and 10 ppm and allows a current to flow between both ends when the gas concentration is outside the above range.

In the examples of FIG. 16 and FIG. 17, a gas type in which a gate threshold voltage shifts in a negative voltage direction depending on the gas concentration is considered as one example, but even in the case of a gas type in which gate threshold voltage shifts in a positive voltage direction, a similar device can be constituted.

### <Another Example of Work Function Type Sensor: Capacitor Type Sensor>

In the examples of the gas sensor chip 1001 described referring to FIGS. 1 to 17, an FET type sensor which is one of work function type sensors is used. Instead of the FET type sensor, a capacitor type sensor, which is also a work function type sensor may be used. In other words, the sensor FET and the reference FET may be replaced by a sensor capacitor (SCAP) and a reference capacitor (FCAP).

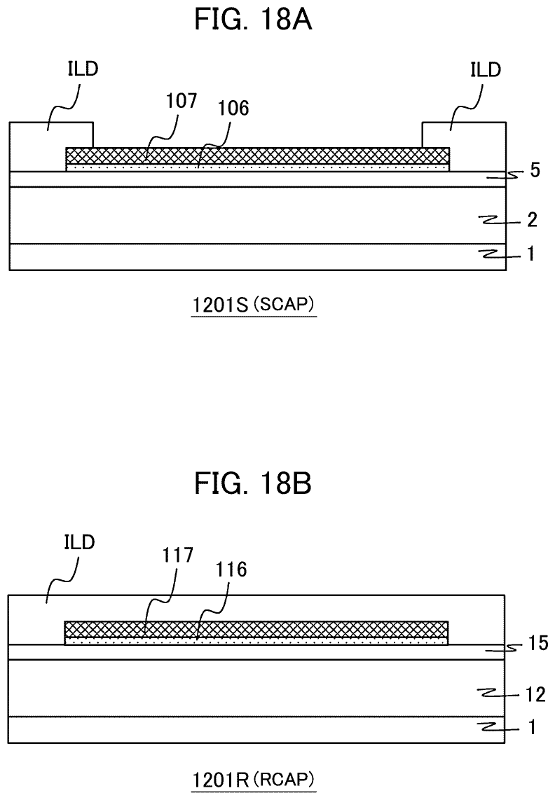

FIG. 18A schematically shows one example of the cross-section of a sensor capacitor. As shown in FIG. 18A, a sensor capacitor 1201S is comprised of a semiconductor substrate 1, a well 2, a gate insulating film 5, and a metal oxide layer 106 and a catalyst metal layer 107, which will be a detection material. The catalyst metal layer 107 of the sensor capacitor 1201S has a surface exposed to the atmosphere containing a detection target gas. For the purpose of fixing or protecting the catalyst metal layer 107, the catalyst metal layer 107 is partially covered with an interlayer insulating film ILD. For the semiconductor substrate 1, for example, silicon, silicon carbide (SiC), or the like can be used.

FIG. 18B schematically shows one example of the cross-section of a reference capacitor. As shown in FIG. 18B, a reference capacitor 1201R has a semiconductor substrate 1, a well 12, a gate insulating film layer 15, and a metal oxide layer 116 and a catalyst metal layer 117 which will be a detection material. The catalyst metal layer 117 of the reference capacitor 1201R has a surface covered with an interlayer insulating film ILD and is separated from the atmosphere containing a detection target gas.

It is to be noted that to the wells 2 and 12 and the catalyst metal layers 107 and 117, a wiring layer made of a metal such as aluminum, tungsten, or platinum is connected to supply power to them from the power sources 1005 to 1008 shown in FIG. 1.

The sensor capacitor 1201S and the reference capacitor 1201R will hereinafter be described as those using N type wells 2 and 12, respectively, but they may use a P type well.

The gas sensor chip 1001 having a constitution using a capacitor type sensor, which is different from that using an FET type sensor, does not need a source drain layer. This simplifies the structure of the gas sensor chip 1001. As will be described later, on the other hand, it is necessary to measure an electrostatic capacity by using an AC voltage not necessary when the FET type sensor is used.

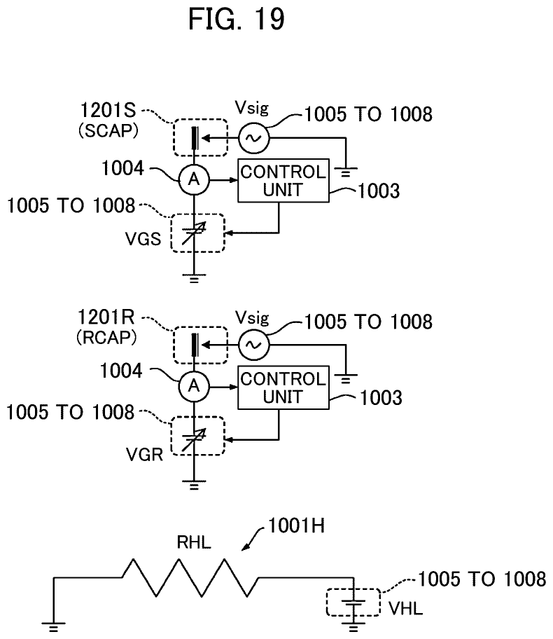

FIG. 19 shows one example of the operation during detection of the concentration of a detection target gas in the gas sensor chip using a capacitor type sensor. A current is applied to a heater wire constituting a heater unit 1001H and by Joule heat generated at a resistor RHL of the heater wire, the sensor capacitor 1201S and the reference capacitor 1201R are heated to a predetermined temperature, for example, 100 °C. To the wells 2 and 12 of the sensor capacitor 1201S and the reference capacitor 1201R, 0 V is applied. A voltage VGR, which is a variable voltage, is applied to the gate of the reference capacitor 1201R from power sources 1005 to 1008, and a variable voltage VGS is applied to the gate of the sensor capacitor 1201S from power sources 1005 to 1008. In addition to the voltages VGR and VGS which are DC voltages, an AC voltage having an amplitude Vsig is applied to the respective terminals of the wells 2 and 12 from the power sources 1005 to 1008. The AC current that flows in the respective gate terminals of the sensor capacitor 1201S and the reference capacitors 1201R is measured using a current detection unit 1004 to detect an electrostatic capacity C (SCAP) of the sensor capacitor 1201S and an electrostatic capacity (RCAP) of the reference capacitor 1201R. The control unit 1003 controls the voltages VGS and VGR, which are variable voltages, so that they coincide with a certain electrostatic capacity C0. A difference VGRS between VGR and VGS at this time is determined as in Equation 1.

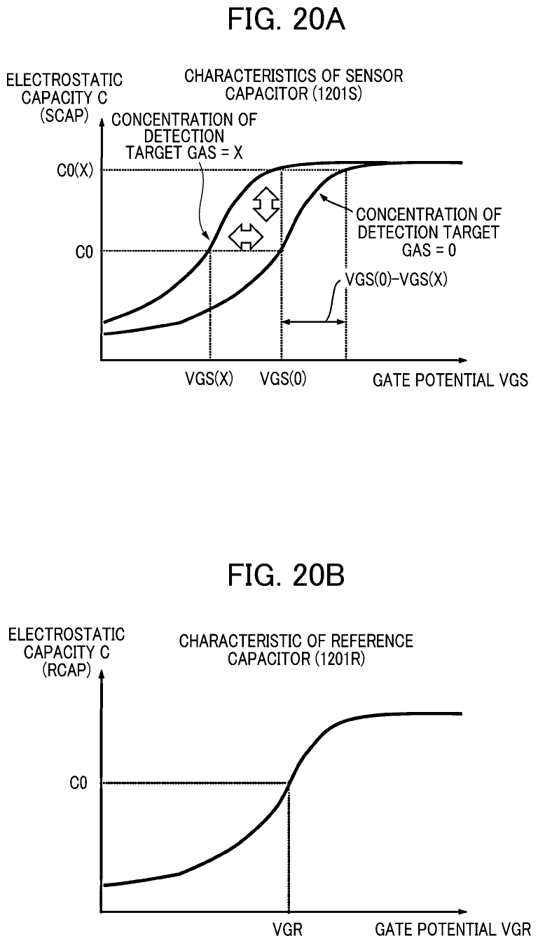

FIG. 20A shows one example of an electrostatic capacity—gate voltage characteristic curve of a sensor capacitor. FIG. 20B shows one example of an electrostatic capacity—gate voltage characteristic curve of a reference capacitor. In the sensor capacitor 1201S, the catalyst metal layer 107 is exposed so that the work function of the catalyst metal layer 7 changes depending on a detection target gas. Between when the concentration of a detection target gas is 0 and when it is X, the electrostatic capacity—gate voltage characteristic curve shows a parallel shift in a voltage direction. As a result, the gate voltage at which the electrostatic capacity is C0 changes from VGS(0) to VGS(X).

On the other hand, since the catalyst metal layer 117 of the reference capacitor 1201R is covered with the interlayer insulating film ILD, even a change in the concentration of the detection target gas does not cause a change in the electrostatic capacity—gate voltage characteristics and the electrostatic capacity value when the voltage VGR is applied to the gate remains constant at C0. As can be understood from the above description, the gas sensor chip 1001 using the sensor capacitor 1201S and the reference capacitor 1201R, similar to that using the sensor FET 1001S and the reference FET 1001R, makes it possible to detect a gas concentration based on the gate threshold voltage shift. By placing a plurality of sensor capacitors 1201S on the gas sensor chip 1001 and using, as a gas detection material layer constituting the gate, different metal oxide layer 106 and catalyst metal layer 107, it is possible to change the sensitivity and selectivity to a gas. It is possible to measure the component pattern of the atmosphere containing a plurality of gas types, as in the case where the sensor FET is used.

### <Further Example of Work Function Type Sensor: Diode Type Sensor>

In the examples of the gas sensor chip 1001 described referring to FIGS. 1 to 17, an FET type sensor is used as a work function type sensor. Instead of the FET type sensor, a diode type sensor may be used as the work function type sensor. In other words, the sensor FET and the reference FET may be replaced by a sensor diode (SDIODE) and a reference diode (FDIODE), respectively.

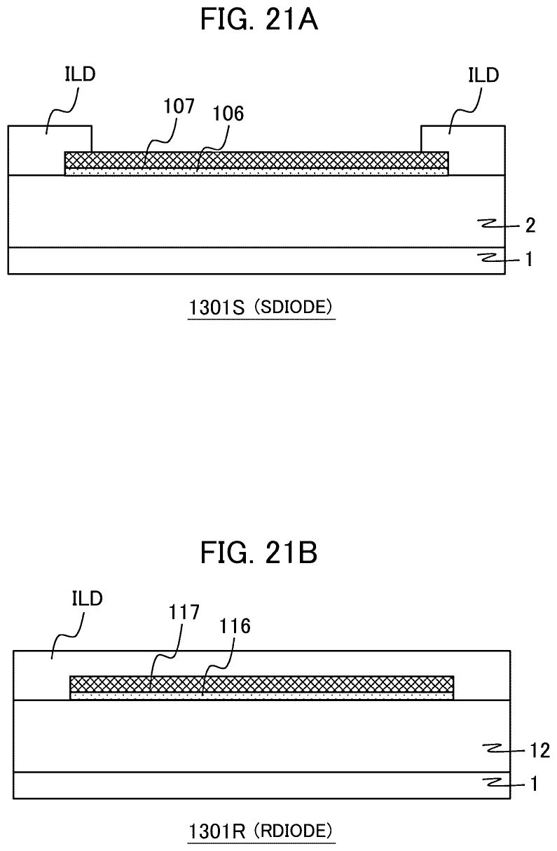

FIG. 21A schematically shows one example of the cross-section of a sensor diode. As shown in FIG. 21A, a sensor diode 1301S has a semiconductor substrate 1, a well 12, and a metal oxide layer 106 and a catalyst metal layer 107 which will be a detection material. The catalyst metal layer 107 of the sensor diode 1301S has a surface exposed to the atmosphere containing a detection target gas and the catalyst metal layer 107 is partially covered with an interlayer insulating film ILD for the purpose of fixing or protecting the catalyst metal layer 107. For the semiconductor substrate 1, for example, silicon, silicon carbide (SiC), or the like can be used.

FIG. 21B schematically shows one example of the cross-section of a reference diode. As shown in FIG. 21B, a reference diode 1301R has a semiconductor substrate 1, a well 12, and a metal oxide layer 116 and a catalyst metal layer 117 which will be a detection material. The catalyst metal layer 117 of the reference diode 1301R has a surface covered with an interlayer insulating film ILD and is separated from the atmosphere containing a detection target gas.

It is to be noted that to the wells 2 and 12 and the catalyst metal layers 107 and 117, a wiring layer made of a metal such as aluminum, tungsten, or platinum is connected to supply power to them from the power sources 1005 to 1008 shown in FIG. 1.

The sensor diode 1301S and the reference diode 1301R will hereinafter be described as those using a N type well as the wells 2 and 12, respectively, but they may use a P type well.

The gas sensor chip 1001 having a constitution using a diode type sensor has a simple structure compared with a constitution using an FET type sensor. An AC voltage which is required for the use of a capacitor type sensor is not necessary.

When the FET type sensor or capacitor type sensor is used, no DC current flows in any of the metal oxide layers 6, 16, 106, and 116 and the catalyst metal layers 7, 17, 107, and 117 which will be a gas detection material. In the constitution using a diode type sensor, on the other hand, a DC current flows in the metal oxide layers 106 and 116 and the catalyst metal layers 107 and 117.

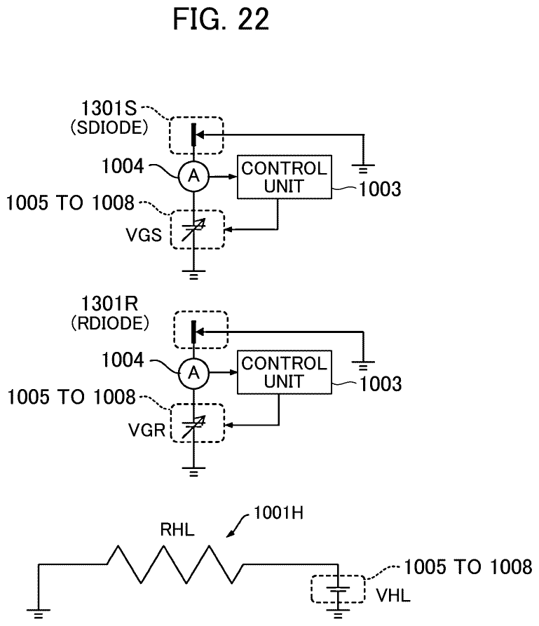

FIG. 22 shows one example of the operation during detection of the concentration of a detection target gas in a gas sensor chip using a diode type sensor. A current is applied to a heater wire constituting a heater unit 1001H and by Joule heat generated at a resistor RHL of the heater wire, the sensor diode 1301S and the reference diode 1301R are heated to a predetermined temperature, for example, 100 °C. Power sources 1005 to 1008 apply 0 V to the wells 2 and 12 of the sensor diode 1301S and the reference diode 1301R. The power sources 1005 to 1008 apply a voltage VGR, which is a variable voltage, to the gate of the reference diode 1301R, and apply a variable voltage VGS to the gate of the sensor diode 1301S. A current detection unit 1004 measures a current that flows in the gate terminal of each of the sensor diode 1301S and the reference diode 1301R. The control unit 1003 controls VGS and VGR so that their currents coincide with a certain IC. A difference VGRS between VGR and VGS at this time is determined as in Equation 1.

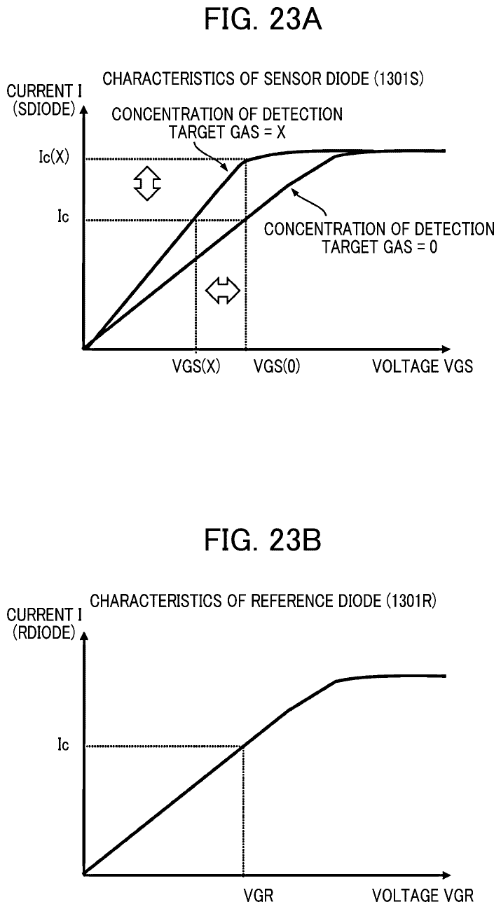

FIG. 23A shows one example of a current—voltage characteristic curve of a sensor diode. FIG. 23B shows one example of a current—voltage characteristic curve of a reference diode. In the sensor diode, the catalyst metal layer 107 is exposed so that the work function of the catalyst metal layer 7 changes depending on a detection target gas. Between the concentration of a detection target gas at 0 and that at X, the current—gate voltage characteristic curve shows a parallel shift in a voltage direction. As a result, the gate voltage at which the current is Ic changes from VGS(0) to VGS(X).

On the other hand, since the catalyst metal layer 117 of the reference diode is covered with the interlayer insulating film ILD, even a change in the concentration of the detection targe gas does not cause a change in the current—gate voltage characteristics and the electrostatic capacity value when a voltage VGR is applied to the gate remains constant at C0. The gas concentration can be detected based on the gate threshold voltage shift, as in the case where the sensor FET and the reference FET are used. By placing a plurality of sensor diodes on the gas sensor chip and using different metal oxide layer 106 and catalyst metal layer 107 as a gas detection material layer, it is possible to change the sensitivity and selectivity to a gas. It is possible to measure the concentration of each gas in the atmosphere containing a plurality of gas types, as in the case where the sensor FET or sensor capacitor is used.

### <Production Method of Gas Sensor Chip>

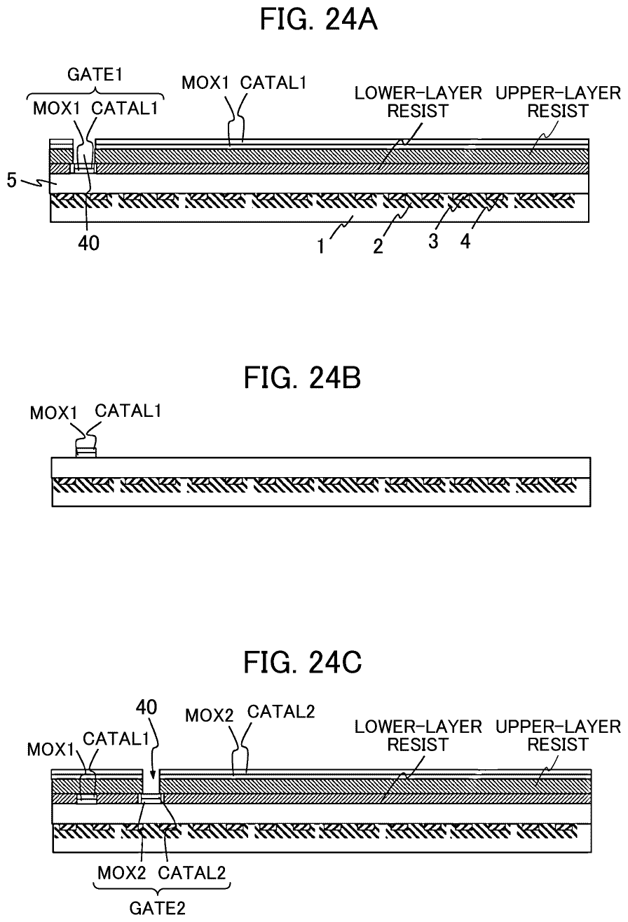

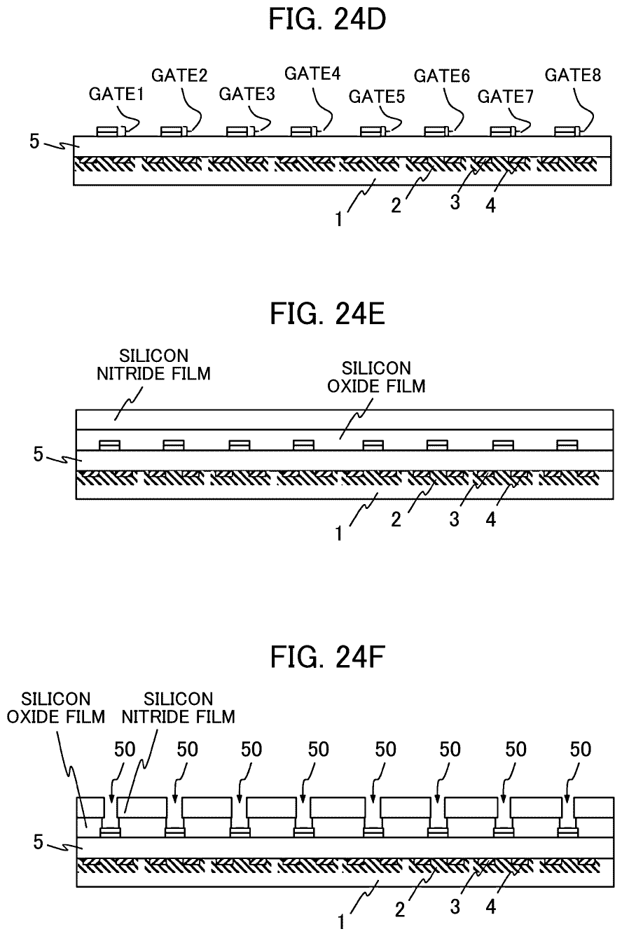

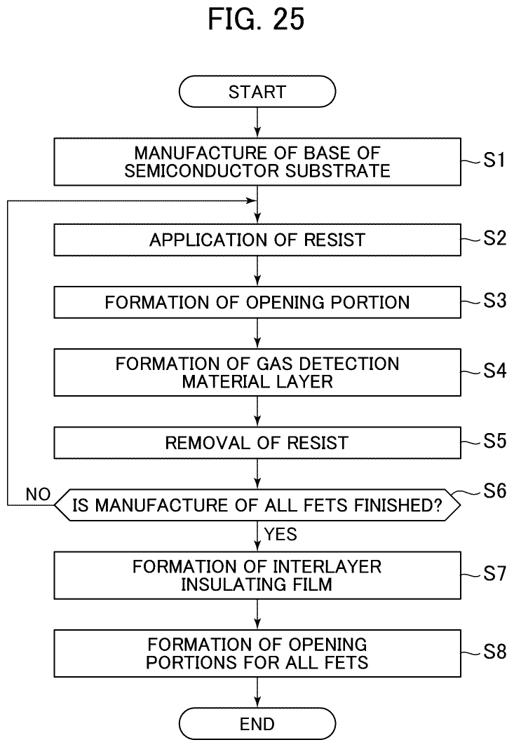

FIGS. 24A to 24F are views for describing one example of a production method of the gas sensor chip of First Embodiment. FIG. 25 is a flow chart showing one example of a production method of the gas sensor chip of First Embodiment. The production method of the gas sensor chip 1001 of First Embodiment is a method of producing, on a semiconductor substrate, plural types of FETs different from each other in gas detection material layer.

The term “plural types of FETs” means FETs used as a sensor FET and a reference FET. This means that when the type of the gas detection material layer of the reference FET and the type of the gas detection material layer of any of the sensor FETs overlap each other and are utilized, the number of FET types to be manufactured is equal to the number of sensor FET types. On the other hand, when the type of the gas detection material layer of the reference FET and the type of the gas detection material layer of the sensor FETs do not overlap each other and are utilized, the number of FET types to be manufactured is equal to the sum of the number of the sensor FET types and the number of reference FET types.

The flow of the production method of a gas sensor chip will be described. As shown in FIG. 25, in Step S1, a treatment of forming a well 2, a source layer 3, and a drain layer 4 on a semiconductor substrate 1 and then forming a gate insulating film 5 on the drain layer is performed. The semiconductor substrate 1 is, for example, a silicon substrate. The gate insulating film 5 includes, for example, an impurity layer and an SiO₂ film. By this step S1, the base of FET is manufactured. It is to be noted that a conventionally-used known method can be used for this step S1.

In Step 2, a treatment of applying a resist on the semiconductor substrate 1 having the base manufactured thereon in Step S1 is performed. The resist may be, for example, two layers of a lower-layer resist and an upper-layer resist, making it possible to form an undercut when it is developed. When the resist is applied, for example, a lift-off process using a resist mask may be used for manufacturing a plurality of sensor FETs different from each other in a gas detection material layer.

In Step S3, on the semiconductor substrate 1 having the resist applied thereto, a treatment of making an opening portion 40 in a region (not limited to one region) in which a catalyst metal layer of some sensor FETs is formed is performed by an exposure step and a development step.

In Step S4, a treatment of forming, on the semiconductor substrate 1, a metal oxide layer and a catalyst metal layer as a gas detection material layer is performed. Step S4 is, as described later, repeated as many times as the number of FET types to be manufactured. During the N-th (N≥1) implementation of Step S4, MOX(N) and CATAL(N) are used as the metal oxide layer and the catalyst metal layer, respectively. For example, during the first implementation of Step S4, a metal oxide layer MOX1 and a catalyst metal layer CATAL1 are formed (FIG. 24A). During the second implementation of Step S4, a metal oxide layer MOX2 and a catalyst metal layer CATAL2 are formed (FIG. 24C). It is to be noted that, for example, the first “metal oxide layer MOX1 and catalyst metal layer CATAL1” from the left in FIG. 24C are one example of a “first gas detection material layer” in the present invention. For example, the second “metal oxide layer MOX2 and catalyst metal layer CATAL2” from the left in FIG. 24C are one example of a “second gas detection material layer” in the present invention.

In Step S5, a treatment of removing the resist thus applied is performed. By the treatment in Step S4, the metal oxide layer MOX1 and the catalyst metal layer CATAL1 are formed also on the upper layer of the upper-layer resist, but the metal oxide layer MOX1 and the catalyst metal layer CATAL1 over the upper layer of the upper-layer resist are also removed by the resist removal in Step S5 (FIG. 24B). As a result, a film stack of the metal oxide layer MOX1 and the catalyst metal layer CATAL1 is formed via the gate insulating film 5 in a channel portion of some sensor FETs.

In Step S6, a treatment of determining whether or not all the FETs planned have been manufactured is performed. In other words, the treatment of determining whether or not the number of manufactured FET types has reached the number of planned types is performed. When it is determined in the above step that all the planned FETs have been manufactured, the step proceeds to Step 7. On the other hand, when it is determined in the above step that all the planned FETs have not been manufactured, the step returns to Step S2 and the treatment from Step S2 to Step S5 is repeated until the number of manufactured FET types reaches the number of planned types.

For example, in the second implementation of Step S2, a treatment of applying a resist on the semiconductor substrate 1 and conducting an exposure step and a development step is performed. In the second implementation of Step S3, a treatment of forming an opening portion 40 in a region, which is different from the place (FIG. 24B) where the film stack of the metal oxide layer MOX1 and the catalyst metal layer CATAL1 has already been formed and in which a catalyst metal layer of some FETs is to be formed, is performed. Then, in the second implementation of Step S4, a treatment of forming a metal oxide layer MOX2 and a catalyst metal layer CATAL2 is performed (FIG. 24C). In the above treatment, the metal oxide layer MOX2 thus formed is made different from MOX1 in any of a material, a film thickness, and a crystal particle size; or the catalyst metal layer CATAL2 thus formed is made different from CATAL1 in any of a material, a film thickness, or a crystal particle size. The metal oxide layer MOX2 and the catalyst metal layer CATAL2 are formed also on the upper layer of the upper-layer resist but by performing the second implementation of Step S5, the resist is removed and at the same time, the metal oxide layer MOX2 and the catalyst metal layer CATAL2 on the upper layer of the upper-layer resist are removed.

By repeating the aforesaid Steps S2 to S5 in order, plural types of gas detection material layers having the catalyst metal layer stacked on the metal oxide layer can be formed (FIG. 24D). In the example of FIG. 24D, an example having eight types of gas detection material layers Gate 1 to Gate 8 is shown, but the number of the types of the gas detection material layers, that is, the number of FET types is of course not limited to this example. By changing the repetition number of Step 2 to Step 5, the types of the gas detection material layers to be formed can be changed. In practice, for example, several types, several hundred types, or several thousand types of gas detection material layers are presumed to be formed.

After formation of the plural required types of gas detection material layers, a treatment of forming a silicon oxide film and a silicon nitride film which will be an interlayer insulating film is performed in Step S7 (FIG. 24E). In the present step, although not shown in the drawing, a treatment of forming a contact hole for supplying power to a source, a drain, a well, and a gate in the aforesaid interlayer insulating film comprised of the silicon oxide film and the silicon nitride film is performed. In addition, a treatment of forming a metal film such as aluminum or tungsten film which will be a wiring and subjecting it to a known lithography technology and a dry etching technology to obtain the wiring is performed. It is to be noted that the term “silicon nitride film” is one example of “an impurity layer” in the present invention.

Next, in Step 8, a treatment of forming an opening portion 50 in the catalyst metal layer for sensor FET in the interlayer insulating film, in other words, in the upper portion of the gas detection material layer is performed. By the above treatment, the catalyst metal layer for the sensor FET is exposed to the atmosphere and thus, a sensor chip is completed (FIG. 24). Although not shown in the drawing, for the manufacture of a reference FET, when the opening portion 50 of the interlayer insulating film is formed on the upper layer of catalyst metal layer for the sensor FET in Step S8 shown in FIG. 24F, the interlayer insulating film is left without performing the treatment of forming an opening in the interlayer insulating film over the upper layer of the catalyst metal layer for reference FET.

In the production method of a gas sensor chip described referring to FIGS. 24A to 24F, a method of making use of a resist mask and a lift-off process for the formation of a gas detection material layer was described as one example. But not only the aforesaid method but also another method such as a method of using a metal mask may of course be used. This will also apply to the following modification examples.

The method of producing a gas sensor chip according to the embodiment of the present invention includes a step of forming an impurity layer on a semiconductor substrate, a step of forming, on the resulting semiconductor substrate, a first gas detection material layer containing a catalyst metal layer, and a step of forming a second gas detection material layer containing a catalyst metal layer at a position different from that of the first gas detection material layer, on the semiconductor substrate. The first gas detection material layer and the second gas detection material layer are different from each other in at least one of the type, film thickness, and crystal particle size of a material constituting the gas detection material layer.

Such a method of producing a gas sensor chip makes it possible to produce a gas sensor chip having plural types of work function type sensors on one semiconductor substrate by a production method similar to that of a MOSFET having a simple structure and at the same time, produce the gas sensor chip at a low cost. As a result, sensing of a gas in the atmosphere can be achieved at a low cost.

In the aforesaid production method of a gas sensor chip described referring to FIGS. 24A to 24F, the gas detection material layers each comprised of the metal oxide layer and the catalyst metal layer are formed one by one by a single step for the individual sensor FETs. As another production method of a gas sensor chip, however, there is also a method of forming the metal oxide layer or the catalyst metal layer which is common to a plurality of sensor FETs at once. Described specifically, steps of forming a plurality of gas detection material layers, for example, a step of forming a first gas detection material layer and a step of forming a second gas detection material layer may be performed after divided into plural times of a film formation step using a mask. The gas detection material layer contains, for example, one or more metal oxide layers and one or more catalyst metal layers, or contains one or more catalyst metal layers without containing a metal oxide layer.

### <Modification Example 1 of Gas Sensor Chip Production Method>

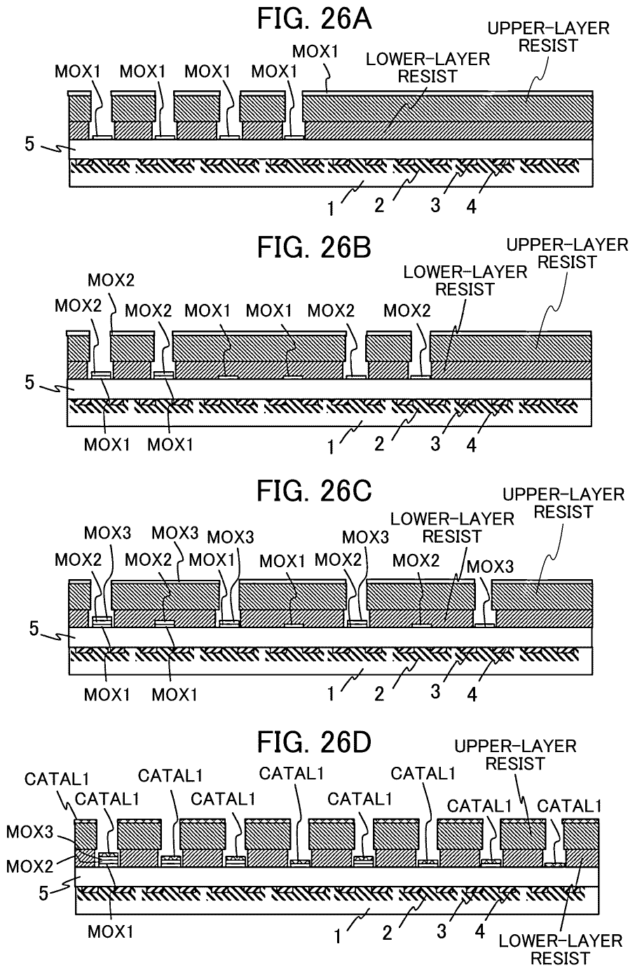

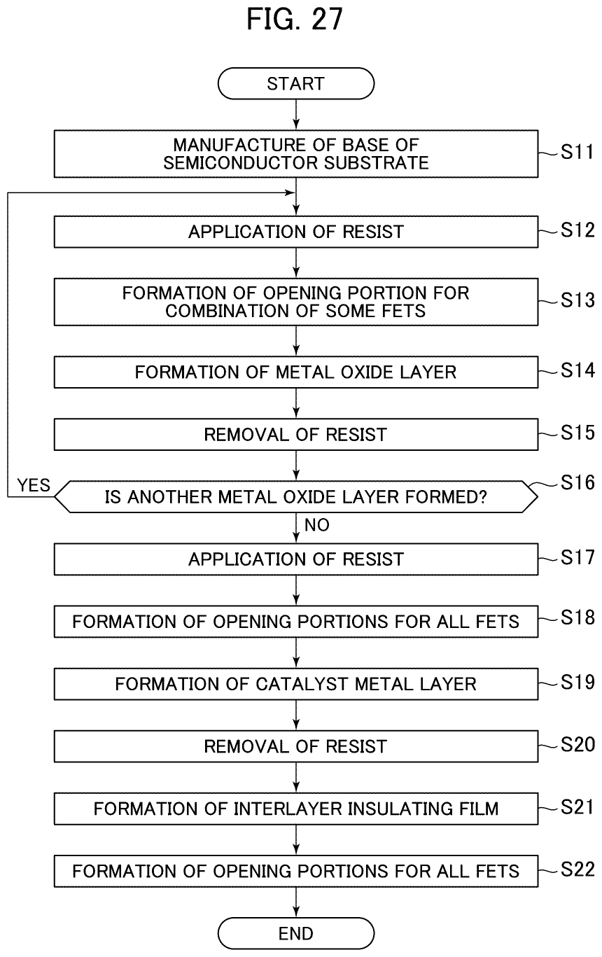

FIGS. 26A to 26D are views for describing Modification Example 1 of the gas sensor chip production method. FIG. 27 is a flow chart showing Modification Example 1 of a gas sensor chip production method.

For example, as shown in FIG. 27, Step Sl1 is performed as in the aforesaid Step S1 and a treatment of forming a well 2, a source layer 3, and a drain layer 4 on a semiconductor substrate 1, forming a gate insulating film 5 on the drain layer, and forming a base is performed.

In Step S12, a treatment of applying a resist to the semiconductor substrate 1 having the base thereon is performed.

In Step S13, a treatment of forming, on the semiconductor substrate 1 having the resist applied thereto, an opening portion at a portion of a position corresponding to the combination of some FETs to be manufactured is performed. In the second and subsequent implementation of Sep S13, an opening portion may be formed at a portion of a position corresponding to the combination of some FETs, among all the FETs to be manufactured, which include FETs for which an opening portion has been formed before, or at a portion of a position corresponding to the combination of FETs for which an opening portion has not been formed before.

In Step S14, a treatment of forming a metal oxide layer, that is, MOX(N) layer on the semiconductor substrate 1 having an opening portion formed therein. The term “N” means the number of implementation times of Step S14 and a metal oxide layer MOX1 is formed by the first implementation of this step and a metal oxide layer MOX2 is formed by the second implementation of this step.

FIG. 26A shows an example of the state in which an opening portion is formed at portions corresponding to the first to fourth FETs from the left on the semiconductor substrate 1 having the resist applied thereto, and the metal oxide layers MOX1s are formed on them, respectively.

In Step S15, a treatment of removing the resist is performed. The metal oxide layer formed on the resist is removed together with the resist.

In Step S16, whether or not another metal oxide layer will be formed is determined. When it is determined that another metal oxide layer will be formed, the step returns to Step S12. When it is determined that another metal oxide layer will not be formed, Step S17 is performed. This means that until the formation of all the metal oxide layers to be formed is finished, the treatment of Steps S12 to S15 are repeated.

FIG. 26B shows an example of the state in which the semiconductor substrate 1 shown in FIG. 26A is subjected to resist removal (Step S15) and resist application (Step S12) successively; opening portions are formed at portions corresponding to the first to second and the fifth to sixth FETs from the left (Step S13), respectively; and the metal oxide layer MOX2 is formed on them (Step S15).

This means that in the example of FIG. 26B, on the semiconductor substrate 1, stacks of the metal oxide layer MOX1 and the metal oxide layer MOX2 are formed at portions corresponding to the first to second FETs from the left, respectively; the metal oxide layers MOX1 are formed at portions corresponding to the third to fourth FETs from the left, respectively; and the metal oxide layers MOX2 are formed at portions corresponding to the fifth to sixth FETs from the left, respectively.

FIG. 26C shows an example of the state in which the semiconductor substrate 1 shown in FIG. 26B is subjected to resist removal (Step S15) and resist application (Step S12) successively; opening portions are formed at portions corresponding to the first, third, fifth, and seventh FETs from the left (Step S13), respectively; and metal oxide layer MOX3 are formed on them (Step S15).

This means that in the example of FIG. 26C, on the semiconductor substrate 1, a stack of the metal oxide layers MOX1, MOX2 and MOX3 is formed at a portion corresponding to the first FET from the left. A stack of the metal oxide layers MOX1 and MOX2 is formed at a portion corresponding to the second FET from the left. A stack of the metal oxide layers MOX1 and MOX3 is formed at a portion corresponding to the third FET from the left. The metal oxide layer MOX1 is formed at a portion corresponding to the fourth FET from the left. A stack of the metal oxide layers MOX2 and MOX3 is formed at a portion corresponding to the fifth FET from the left. The metal oxide layer MOX2 is formed at a portion corresponding to the sixth FET from the left. The metal oxide layer MOX3 is formed at a portion corresponding to the seventh FET from the left. A metal oxide layer is not formed at a portion corresponding to the eighth FET from the left.

In Step S17, a treatment of applying a resist to the semiconductor substrate 1 is performed.

In Step S18, a treatment of forming opening portions at portions corresponding to all the FETs to be manufactured is performed, respectively, on the semiconductor substrate having the resist applied thereto.

In Step S19, a treatment of forming a catalyst metal layer CATAL1, which will be a gate, on the semiconductor substrate 1 having the opening portions formed thereon is performed. It is to be noted that the first gas detection material layer from the left in FIG. 26D, that is, “metal oxide layers MOX1” to “MOX3” and “catalyst metal layer CATAL1” is one example of the “first gas detection material layer” in the present invention. For example, the eighth gas detection material layer from the left in FIG. 26D, that is, “catalyst metal layer CATAL1” is one example of the “second gas detection material layer” in the present invention.

In Step S20, a treatment of removing the resist is performed. The catalyst metal layer CATAL1 formed on the resist is removed together with the resist.

In Step S21, a treatment of forming a silicon oxide film and a silicon nitride film serving an interlayer insulating film is performed.

In Step S22, a treatment of forming, on the semiconductor substrate 1, an opening portion at a position portion corresponding to all the FETs whose manufacture is planned is performed

Although not shown in the drawings, when a reference FET is manufactured, the interlayer insulating film on the catalyst metal layer of the reference FET is left without forming an opening portion in the film, at the time when an opening portion 50 is formed in the interlayer insulating film at the gate portion of FETs. The aforesaid treatment for manufacturing the reference FET is also applied to the modification examples of the production method of a gas sensor chip shown below.

According to Modification Example 1 of such a production method of a gas sensor chip, similar to the aforesaid production method, a gas chip sensor having plural types of sensor FETs formed therein can be produced.

For example, it is possible to increase the number of the types of sensor FETs having gas detection material layers different from each other, by forming the metal oxide layer MOX2 for some of the FETs, among the FETs which are the same in aspect in whether or not the metal oxide layer MOX1 has been formed in a structure before formation of the metal oxide layer MOX2, and not forming the metal oxide layer MOX2 for the remaining FETs, as shown in the example of FIG. 26B. This is because the structure of the gas detection material layer is the same before formation of the metal oxide layer MOX2, but it changes to another one as the result of the formation of the metal oxide layer MOX2.

Similarly, it is possible to increase the number of the types of sensor FETs having gas detection material layers different from each other by forming the metal oxide layer MOX3 for some of the FETs, among the FETs which are the same in stack structure comprised of the metal oxide layer MOX1 and the metal oxide layer MOX2 before formation of the metal oxide layer MOX3, and not forming the metal oxide layer MOX3 for the remaining FETs, as shown in the example of FIG. 26C.

Although not shown also in this drawing, when a reference FET is manufactured, the interlayer insulating film on the upper layer of the gate portion of the reference FET is left without forming an opening portion in the film, at the time when an opening portion 50 is formed in the interlayer insulating film at the gate portion of FETs.

### <Modification Example 2 of Gas Sensor Chip Production Method>

In Modification Example 1 of the gas sensor chip production method, the catalyst metal layer is the same in all the FETs (CATAL1) It is however possible to form the catalyst metal layer while dividing it into plural types. Modification Example 2 of the gas sensor chip production method will hereinafter be described.

It is to be noted that in the following description of each Modification Example, a description on the step of applying and removing a resist, the step of forming an opening portion in a resist layer, and the step of forming an interlayer insulating film will be omitted unless otherwise necessary in order to simplify the description. Similarly, descriptions on the step of forming an opening portion in an interlayer insulating film on the upper layer of the gate portion of a sensor FET, and leaving the interlayer insulating film over the upper layer of the gate portion of the reference FET without forming an opening portion in the film are omitted unless otherwise necessary.

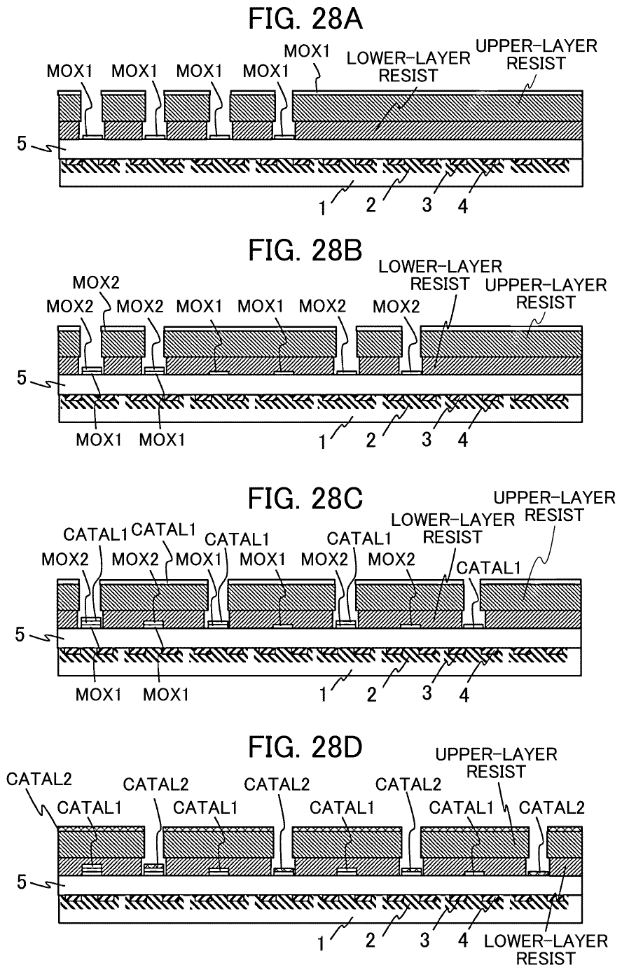

FIGS. 28A to 28D are views for describing Modification Example 2 of the gas sensor chip production method. For example, on the semiconductor substrate 1 on which a well 2, a source layer 3, a drain layer 4, and a gate insulating film 5 have been formed to form a base, a treatment of forming a metal oxide layer MOX1 for the combination of some of FETs to be manufactured is performed (FIG. 28A). In addition, a treatment of forming a metal oxide layer MOX2 for another combination of some FETs (FIG. 28B). Next, a treatment of forming a catalyst metal layer CATAL1 which will be a gate is performed for some FETs (FIG. 28C) and a treatment of forming a catalyst metal layer CATAL2 which will be a gate is performed for the remaining FETs (FIG. 28D).

As shown in the example of FIG. 28B, a metal oxide layer MOX2 is formed for some of the FETs, among the FETs which are the same in aspect in whether or not the metal oxide layer MOX1 has been formed in a structure before formation of the metal oxide layer MOX2, and the metal oxide layer MOX2 is not formed for the remaining FETs. Then, the number of the types of the sensor FETs having gas detection material layers different from each other can be increased. This is because the structure of the gas detection material layers which are the same before formation of the metal oxide layer MOX2 becomes a different one by forming the metal oxide layer MOX2.

As shown in the examples of FIGS. 28C and 28D, a catalyst metal layer CATL1 is formed for some of the FETs, among the sensor FETs which are the same in stack structure comprised of the metal oxide layer MOX1 and the metal oxide layer MOX2 before formation of catalyst metal layers CATAL1 and CATAL2, and a catalyst metal layer CATAL2 is formed for the remaining FETs. Then, the number of the types of the sensor FETs different in gas detection material layer can be increased. This is because the structures of the gas detection material layers which are the same before formation of the catalyst metal layers CATAL1 and CATAL2 become different ones due to the formation of the catalyst metal layers CATAL1 and CATAL2.

### <Modification Example 3 of Gas Sensor Chip Production Method>

Modification Examples 1 and 2 of the gas sensor chip production method each include a step of stacking metal oxide layers one after another. In these Examples, however, the catalyst metal layer is comprised of a single CATAL1 layer or a single CATAL2 layer and they do not include a step of stacking catalyst metal layers one after another. It is however possible to increase the number of the types of the gas detection materials of sensor FETs by adding a step of stacking catalyst metal layers one after another to the step of stacking metal oxide layers one after another. Modification Example 3 of the gas sensor chip production method will hereinafter be described. Here, descriptions on the resist application and removal steps and the step of forming an opening portion are omitted to simplify the description.

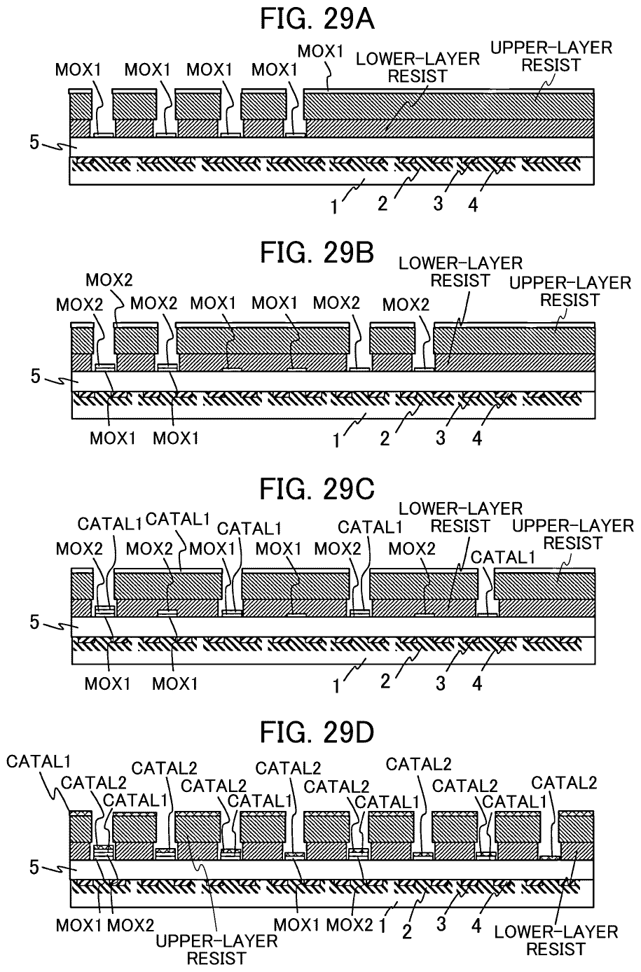

FIG. 29A to FIG. 29D are each a view for describing Modification Example 3 of the gas sensor chip production method. For example, on the semiconductor substrate 1 on which a well 2, a source layer 3, a drain layer 4, and a gate insulating film 5 are formed to manufacture a base, a treatment of forming a metal oxide layer MOX1 for the combination of some of FETs to be manufactured is performed (FIG. 29A). In addition, a treatment of forming a metal oxide layer MOX2 for another combination of some of them is performed (FIG. 29B). Next, a treatment of forming a catalyst metal layer CATAL1 which will be a gate for some FETs is performed (FIG. 29C). Next, a treatment of forming a catalyst metal layer CATAL2 which will be a gate is performed for all the FETs including the FETs having the CATAL1 formed thereon (FIG. 29D).

A treatment of forming the catalyst metal layer CATAL1 is performed as shown in FIG. 29C for some FETs, among FETs which are the same in a stack structure of the metal oxide layer MOX1 and the metal oxide layer MOX2 before formation of the catalyst metal layer CATAL1. A treatment of forming the catalyst metal layer CATAL2 is performed for the remaining FETs. By such a treatment, the number of the types of the sensor FETs different in gas detection material layer can be increased. This is because the structures of the gas detection material layers which are the same before formation of the catalyst metal layer CATAL1 become different ones by the formation of the catalyst metal layer CATAL1.

### <Modification Example 4 of Gas Sensor Chip Production Method>

In Modification Examples 1 to 3 of the gas sensor chip production method, the metal oxide layer has already been a metal oxide film at the time of film formation, but it is also possible to form a metal layer at the time of film formation and in a subsequent step, the metal layer is oxidized into a metal oxide layer.

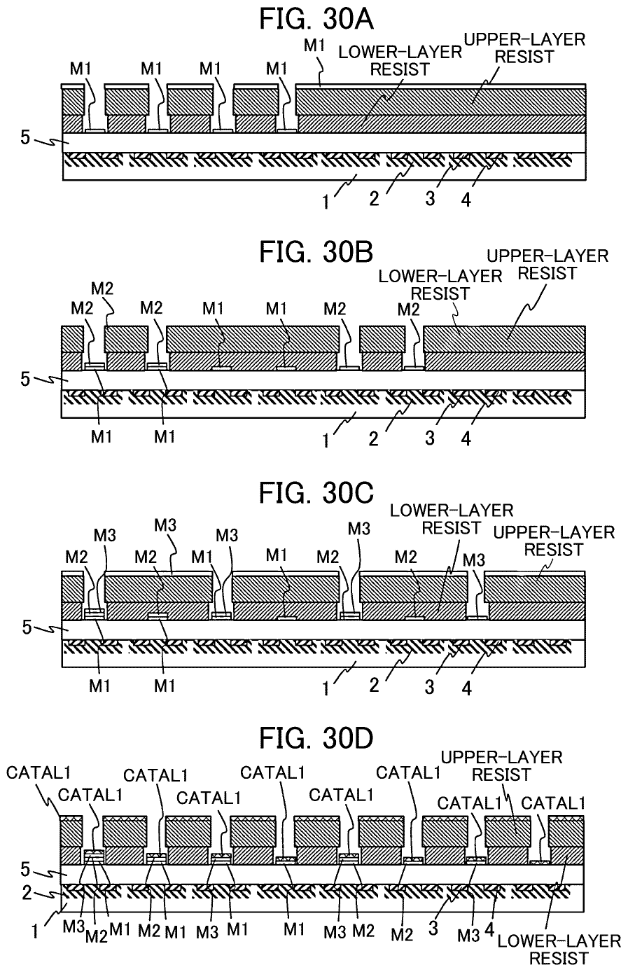

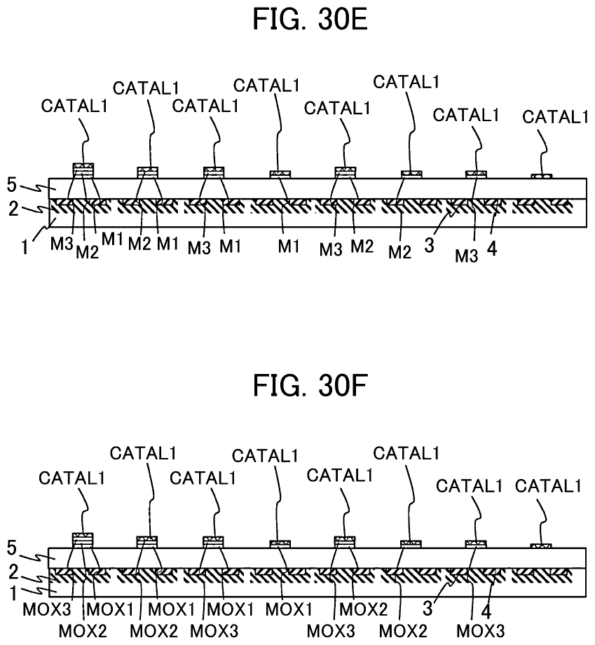

FIGS. 30A to 30F are views for describing Modification Example 4 of the gas sensor chip production method. For example, on a semiconductor substrate 1 on which a well 2, a source layer 3, a drain layer 4, and a gate insulating film 5 are formed to manufacture a base, a treatment of forming a metal layer M1 which will be a metal oxide layer MOX1 in a later oxidation step is performed for the combination of some of FETs to be manufactured (FIG. 30A). A treatment of forming a metal layer M2 which will be a metal oxide layer MOX2 in the later oxidation step is performed for another combination of some of them (FIG. 30B). A treatment of forming a metal layer M3 which will be a metal oxide layer MOX3 in the later oxidation step is performed for a further combination of some of them (FIG. 30C). Then, a treatment of forming a catalyst metal layer CATAL1 which will be a gate is performed for all the FETs (FIG. 30D).

A treatment of removing a resist is performed for the semiconductor substrate 1 having the aspect shown in FIG. 30D (30E). Then, annealing is performed at about 400 °C in the air to convert the metal layers M1, M2, and M3 into the metal oxide layers MOX1, MOX2, and MOX3, respectively (FIG. 30F). When the metal layers M1, M2, and M3 are oxidized, volume expansion thereof occurs. By appropriately selecting the type and film thickness of the metal layers M1, M2, and M3 and the catalyst metal layer CATAL1, expanded MOX1, MOX2, and MOX3 enter the crystal grain boundary of the uppermost catalyst metal layer CATAL1, making it possible to form a nanostructure of the catalyst metal layer CATAL1. The nanostructure of the catalyst metal layer CATAL1 is an important structure for improving sensitivity of a gas sensor.

By forming the metal layer M2 for some of the FETs, among FETs which are the same in aspect in whether or not the metal layer 1 has been formed on the semiconductor substrate 1 before formation of the metal layer M2, and not forming the metal layer M2 for the remaining FETs, as shown in FIG. 30, it is possible to increase the number of the types of sensor FETs having gas detection material layers different from each other. This is because the structures of gas detection material layers which are the same before formation of the metal layer M2 become different ones by the formation of the metal layer M2. Similarly, by forming the metal layer M3 for some of the FETs, among FETs which are the same in stack structure comprised of the metal layer M1 and the metal layer M2 on the semiconductor substrate 1 before formation of the metal layer M3, and not forming the metal layer M3 for the remaining FETs, as shown in FIG. 30C, it is possible to increase the number of the types of sensor FETs different in gas detection material layers.

### <Modification Example 5 of Gas Sensor Chip Production Method>

Suppose an example of forming a film of a mixed crystal system material such as YSZ (yttria stabilized zirconia) as a special example. The composition formula of YSZ may be (ZrO₂)_{1-z}(Y₂O₃)_{z}. For example, N times film formation is performed to alternately form a ZrO₂ film and a Y₂O₃ film and the thicknesses (number of moles per unit area) of the films thus obtained by this N times film formation are all changed. By this method, 2^{N} pieces of sensor FETs can be formed so as to be different from each other in composition Z of YSZ, that is, different in gas detection material. The sensitivity or selectivity of a sensor changes by a difference in gas detection material, making it possible to detect various gases.

In addition, CeO₂ and Gd₂O₃ can be used as a mixed crystal and it can be indicated by the following composition formula: (CeO₂)_{1-z}(Gd₂O₃)_{z}. For example, N times film formation is performed to alternately form a CeO₂ film and a Gd₂O₃ film and the thicknesses (number of moles per unit area) of the films thus obtained by this N times film formation are all changed. By this method, 2^{N} pieces of sensor FETs can be formed so as to be different from each other in the composition Z of (CeO₂)_{1-z}(Gd₂O₃)_{z}, that is, different in gas detection material. The sensitivity or selectivity of a sensor changes by a difference in gas detection material, making it possible to detect various gases.

In addition, BaCe_{1-z}Y_{z}O₃, BaZr_{1-z}Y_{z}O₃, SrCe_{1-z}Y_{z}O₃, and SrZr_{1-z}Y_{z}O₃ can be used as a mixed crystal. When BaCe_{1-z}Y_{z}O₃ is used, for example, N times film formation is performed to alternately form a BaCeO₃ film and a BaYO_{3-δ}, film and the thicknesses (number of moles per unit area) of the films thus obtained by this N times film formation are all changed. Thus, 2^{N} pieces of sensor FETs can be formed so as to be different from each other in the composition Z of BaCe_{1-z}Y_{z}O₃, that is, different in gas detection material. The sensitivity or selectivity of a sensor changes by a difference in gas detection material, making it possible to detect various gases. This also applies to BaZr_{1-z}Y_{z}O₃, SrCe_{1-z}Y_{z}O₃, and SrZr_{1-z}Y_{z}O₃.

### <Modification Example 6 of Gas Sensor Chip Production Method>

In the gas sensor chip production method and Modification examples thereof described above, the metal oxide layer or the metal layer which will be a metal oxide layer, and the catalyst metal layer are formed separately. The metal oxide layer doped with a catalyst metal such as platinum, palladium, iridium, rhodium, or ruthenium may also be functioned as the gate of FET. These catalyst metals may be functioned as a catalyst metal layer when their resistance can be reduced to such an extent that the metal oxide layer imparted with electroconductivity can be functioned as a gate. In other words, the metal oxide layer may be included in the catalyst metal layer.

In Modification Example 4 of the gas sensor chip production method described above, it is possible to dope the metal layers M1, M2, and M3, which participate in a subsequent step and will be a metal oxide, with a catalyst metal such as platinum, palladium, iridium, rhodium, or ruthenium. In the oxidation step as shown in the example of FIG. 30F, the metal layers M1, M2, and M3 become the metal oxide layers MOX1, MOX2, and MOX3, respectively and they each be a layer containing the catalyst metal used for doping. These catalyst metals may be functioned as a catalyst metal layer when their resistance can be reduced to such an extent that the metal oxide layer imparted with electroconductivity can be functioned as a gate. In other words, the metal oxide layer may be included in the catalyst metal layer.

### <Effect of Modification Examples of Gas Sensor Chip Production Method>

In Modification Examples 1 to 6 of the gas sensor chip production method described above, the number of the types of the gas detection material layer can be increased to be twofold at most whenever the material layer is formed. By N+1 times film formation of the material, 2^{N} types of gas detection material layers can be formed, because the catalyst metal layer is necessary for all the sensor FETs. When the number of the types of the sensor FETs necessary on the gas sensor chip is particularly large, the method of Modification Examples 1 to 6 of the gas sensor chip production method can exponentially reduce the number of production steps of the gas sensor chip. For example, the gas sensor chip production method described referring to FIGS. 24A to 24F need 1024 lithography steps for forming 1024 gas detection materials different in type, but the methods of Modification Examples 1 to 6 of the gas sensor chip production method each need only 11 lithography steps.

### <Sensing System Using the Gas Sensor of First Embodiment>

As described in the example of FIG. 8B, when the number N of the types of the detection materials of the sensor FET mounted on the gas sensor chip and the number M of the types of the gas components contained in the atmosphere satisfy the following equation: M≤N, the X_Gas1 to X_GasM can be estimated by minimizing the ΔVg_Sum of (Equation 4) for example. When N<M, on the other hand, it is impossible to estimate the X_Gas1 to X_GasM.

However, since the sensitivity or selectivity of the sensor FET to a detection target gas can be changed by changing the temperature of the gas sensor chip, it becomes possible to regard the same sensor FET as a new sensor FET different therefrom in properties by changing the temperature. Even if N pieces of sensors on the same gas sensor chip are used, when measurement is conducted at two temperatures T1 and T2, it is possible to obtain the information equal to that obtained by the measurement using 2^{N} pieces of respectively different sensor FETs. When M≤2N even if N<M, the X_Gas1 to X_GasM can be estimated by minimizing the ΔVg_Sum (T1, T2), which is defined by the following equation 5, relative to the 2^{N} gate threshold voltage shifts.

$$Δ Vg\_Sum ( T 1 , T 2 ) = \qquad ( Equation 5 )$$

$${\{ Δ Vg\_Gate1 ( T 1 ) - Δ Vg\_Gate1 \_Calc ( T 1 ) \}}^{2} +$$

$${\{ Δ Vg\_Gate2 ( T 1 ) - Δ Vg\_Gate2 \_Calc ( T 1 ) \}}^{2} +$$

$$…$$

$${\{ Δ Vg\_GateN ( T 1 ) - Δ Vg\_GateN \_Calc ( T 1 ) \}}^{2} +$$

$${\{ Δ Vg\_Gate1 ( T 2 ) - Δ Vg\_Gate1 \_Calc ( T 2 ) \}}^{2} +$$

$${\{ Δ Vg\_Gate2 ( T 2 ) - Δ Vg\_Gate2 \_Calc ( T 2 ) \}}^{2} +$$

$$…$$

$${\{ Δ Vg\_GateN ( T 2 ) - Δ Vg\_GateN \_Calc ( T 2 ) \}}^{2}$$

Equation 5 shows an example of measuring at two temperatures and this equation can be generalized. When P pieces of gas sensor chips having N pieces of sensor FETs different in gas detection material layer are prepared and these gas sensor chips are used at different temperatures T1 to TP to measure the gas component in the atmosphere; and M≤N×P, X_Gas1 to X_GasM can be estimated.

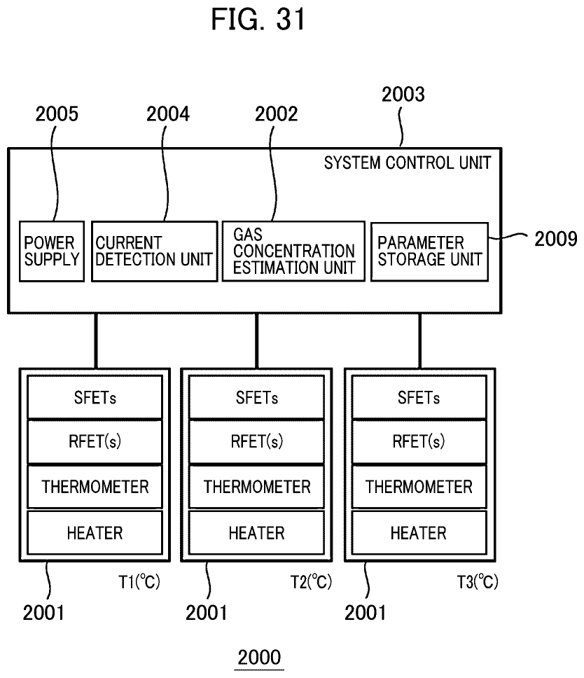

FIG. 31 is a view showing an example of a gas sensing system using a plurality of gas sensor chips at respectively different temperatures. The gas sensing system 2000 shown in FIG. 31 has a plurality of gas sensor chips 2001 and a system control unit 2003 that controls them. In the gas sensor chips 2001, a gas detection material layer has thereon a plurality of sensor FETs different from each other. The gas sensor chips 2001 each has therein a heater unit (Heater), for example, a heater wire capable of individually elevating the temperature of the gas sensor chips 2001. In short, the gas sensing system 2000 has a plurality of gas sensor chips 2001 and a plurality of heater units. The gas sensor chips 2001 has therein a thermometer unit (Thermometer) capable of temperature measurement, for example, a diode. The system control unit 2003 is obtained by systemizing the circuit unit 1100 of FIG. 1 to enable control of a plurality of gas sensors. The system control unit 2003 is, similar to the control unit 1003 of FIG. 1, equipped with a gas concentration estimation unit 2002, a current detection unit 2004, a power source 2005, and a parameter storage unit 2009.

### <Effect of a Gas Sensing System Using a Plurality of Gas Sensor Chips at Different Temperatures>

By using the gas sensing system as shown in FIG. 31, the gas component in the atmosphere can be measured at a plurality of temperatures, for example, three temperatures T1 to T3 °C in the example of FIG. 31. Supposing that the number of gas sensor chips is NC and the following equation M≤N×NC is satisfied, the X_Gas1 to X_GasM can be estimated. The temperatures T1, T2, and T3 can be set, for example, at 100 °C, 200 °C, and 300 °C, respectively.

### <About Modification Examples of the Present Invention>

The present invention is not limited to the aforesaid embodiments but includes various modification examples. For example, the aforesaid embodiments are for describing the invention in detail to facilitate understanding and they are not always limited to have all the constitutions described above. A portion of the constitution of one embodiment may be replaced by the constitution of another embodiment, or to the constitution of one embodiment, the constitution of another embodiment may be added. A portion of the composition of each embodiment may be added with another constitution, deleted, or replaced by another constitution.

### <Supplementary Note>

The present application discloses an inexpensive gas sensor chip in which a plurality of gas sensor elements different in sensitivity and selectivity are assembled on a semiconductor substrate and which can measure a component amount of a plurality of gas types contained in the atmosphere. For example, by forming, on the impurity layer on the semiconductor substrate, gas detection material layers different from each other in at least one of the type, film thickness, and crystal particle size of the material to form a plurality (N pieces) of work function type sensors (for example, sensor FETs) different in sensitivity and selectivity to a gas. Based on the measurement results of the amount of a change of electrical characteristics of N pieces of sensors in the atmosphere, the component amount of each gas of 1 to M types (M≤N) of gases can be measured and for example, the atmospheres different in gas type and the concentration of each gas type can be distinguished.

One example of the gas sensor according to the embodiment of the present application has a plurality of catalyst metal gate FET type sensor elements which have, on a gate thereof, a gas detection material layer which is formed on a semiconductor substrate and is comprised of at least one metal oxide layer different from each other in type, film thickness, or polycrystal particle size of the material or at least one catalyst metal layer different from each other in type, film thickness or polycrystal particle size of the material. With respect to the metal oxide layer and the catalyst metal layer, the catalyst metal layer is formed on the metal oxide layer and is exposed to the atmosphere. The catalyst metal gate FET type sensors may be formed using either one of an N type or a P type. Alternatively, it may be formed using both the N type and P type. Of the catalyst metal gate FET type sensors in an array, a plurality of them may be used in combination to form a catalyst metal gate FET type sensor circuit. A selection transistor for accessing a specific sensor from an array comprised of a plurality of catalyst metal gate FET type sensors may be formed separately from the catalyst metal gate FET type sensors by using a typical gate material that does not react with a gas. Further, one or more reference FETs may be loaded which have, as the catalyst metal layer and metal oxide layer of the gate layer thereof, same as those of any of the aforesaid catalyst metal gate FET type sensors and at the same time, have a gate covered with a protection film so as not to be exposed to the atmosphere. It is also possible to load, on the semiconductor substrate, a heater wire for heating the catalyst metal gate FET type sensor element to control the temperature or a diode element which will be a thermometer for estimating the temperature of the catalyst metal gate FET type sensor element. Power supply to the catalyst metal gate FET type sensor element, the reference FET, the heater wire, or the diode can be performed from an outside power source via a wire bond or by using a TSV technology that supplies power via a hole penetrating the substrate.

One example of the embodiment of the present invention will hereinafter be described.

### [Supplementary Note 1]

A gas sensor chip includes: a semiconductor substrate; and a plurality of gas detection material layers formed at respectively different positions on the semiconductor substrate. The gas detection material layers have a metal oxide layer and a catalyst metal layer formed over the metal oxide layer, the catalyst metal layer being exposed to the atmosphere, and the gas detection material layers are different from each other in the combination of the type, film thickness, and crystal particle size of material of the metal oxide layer and the type, film thickness, and crystal particle size of material of the catalyst metal layer.

### [Supplementary Note 2]

In the gas sensor chip described in Supplementary note 1, with respect to each of the plurality of gas detection material layers, a plurality of sensor capacitors is constituted by using the gas detection material layers as a gate layer and providing a gate insulating film layer and a well between the metal oxide layer and the semiconductor substrate.

### [Supplementary Note 3]

In the gas sensor chip described in Supplementary note 1, with respect to each of the plurality of gas detection material layers, a plurality of sensor diodes is constituted by using the gas detection material layers as a gate layer and providing a well between the metal oxide layer and the semiconductor substrate.

### [Supplementary Note 4]

The gas sensor chip described in Supplementary note 1, further includes a heater wire capable of elevating the temperature of the gas sensor chip from the environmental temperature by supplying power via an electrode terminal.

### [Supplementary Note 5]

The gas sensor chip described in Supplementary note 1, further includes a diode capable of measuring the temperature of the gas sensor chip by measuring an electric current via an electrode terminal.

### LIST OF REFERENCE SIGNS

1: semiconductor substrate
2, 12: well
3, 13: source layer
4, 14: drain layer
5, 15: gate insulating film layer
6, 16, 106: metal oxide layer
7, 17, 107: catalyst metal layer
40: opening portion of resist
50: opening portion of interlayer insulating film
1000, 2000: gas sensing system
1001, 2001: gas sensor chip
1001H; heater unit (Heater)
1001R: reference FET (RFET)
1001S: sensor FET (SFET)
1001SA: sensor FET array
1001T: thermometer unit (Thermometer)
1002, 2002: gas concentration estimation unit
1003: control unit
2003: system control unit
1004, 2004: current detection unit
1005, 1006, 1007, 1008, 2005: power source
1009, 2009: parameter storage unit
1010: I/O unit
1020: semiconductor device
1201S: sensor capacitor (SCAP)
1201R: reference capacitor (RCAP)
1301S: sensor diode (SDIODE)
1301R: reference diode (RDIODE)
ILD: interlayer insulating film
X, X₀: gas concentration
X_Gas1, S_GasM: gas concentration
RHL: resistor of heater wire
VD: drain voltage
VSS: voltage of source, well
Ic, Ic(X): current
VGS, VGS(X), VGS(O), VGS(X, Y): voltage
VGR, VGRS: voltage
A: ammeter
ID(SFET): drain current of sensor FET
ID(RFET): drain current of reference FET
IS(SFET): source current of sensor FET
C0: electrostatic capacity
C(SCAP): electrostatic capacity of sensor capacitor
C(RCAP): electrostatic capacity of reference capacitor
I(SDIODE): current of sensor diode
I(RDIODE): current of reference diode
ΔVg, ΔVgs(X): threshold voltage shift of gas sensor
ΔVgmax: maximum value of threshold voltage shift of gas sensor
ΔVg_Gate1, ΔVg_Gate5, ΔVg_GateN: found value of threshold voltage
shift of gas sensor
ΔVg_Gate1_Calc, ΔVg_GateN_Calc: calculated value of threshold voltage shift of gas sensor
Vsig: amplitude of AC voltage
MOX1, MOX2, MOX3: metal oxide layer
CATAL1, CATAL2: catalyst metal layer
M1, M2, M3: metal layer
Z: composition

1. A gas sensor chip, comprising:
a semiconductor substrate; and
a plurality of gas detection material layers formed at respectively different positions on the semiconductor substrate,
wherein the gas detection material layers are configured to have a metal oxide layer and a catalyst metal layer formed over the metal oxide layer, the catalyst metal layer being exposed to the atmosphere, and
the gas detection material layers are different from each other in the combination of the type, film thickness, and crystal particle size of material of the metal oxide layer and the type, film thickness, and crystal particle size of material of the catalyst metal layer.

2. The gas sensor chip according to claim 1,
wherein each of the gas detection material layers is used as a gate layer and a gate insulating film layer, a drain layer, a source layer, and a well are provided between the metal oxide layer and the semiconductor substrate to constitute a plurality of sensor FETs.

3. The gas sensor chip according to claim 2, further comprising, in addition to the sensor FETs, a first FET having a gate layer constituted to be separated from the atmosphere.

4. The gas sensor chip according to claim 2,
wherein some of the sensor FETs are N type FETs and the other sensor FETs are P type FETs.

5. The gas sensor chip according to claim 2,
wherein the sensor FETs respectively have silicon oxide films between the semiconductor substrate and the gate layer, and
the silicon oxide films of the sensor FETs are formed to have plural types of thicknesses.

6. The gas sensor chip according to claim 2,
wherein the sensor FETs include an element having the sensor FETs connected in parallel or in series each other.

7. The gas sensor chip according to claim 2,
wherein the sensor FETs each include a source terminal, a drain terminal, a well terminal, and a gate terminal, and
at least one of the source terminal and the drain terminal is connected to a power supply terminal to the gas sensor chip from the outside via a second FET having a gate layer separated from the atmosphere.

8. The gas sensor chip according to claim 2,
wherein in at least some of the sensor FETs, at least one of the metal oxide layer and the catalyst metal layer has a plurality of layers.

9. The gas sensor chip according to claim 2,
wherein in at least some of the sensor FETs, the metal oxide layer is doped with a catalyst metal or the catalyst metal layer is doped with a metal oxide.

10. A gas sensing system, comprising:
a gas sensor chip;
a heater unit that heats the gas sensor chip to elevate a temperature thereof; and
a control unit that controls the gas sensor chip and the heater unit,
wherein the gas sensor chip includes:
a semiconductor substrate; and
a plurality of gas detection material layers formed at respectively different positions on the semiconductor substrate,
the gas detection material layers are configured to have a metal oxide layer and a catalyst metal layer formed over the metal oxide layer, the catalyst metal layer being exposed to the atmosphere,
the gas detection material layers are different from each other in a combination of a type, a film thickness, and a crystal particle size of the metal oxide layer and a type, a film thickness, and a crystal particle size of the catalyst metal layer, and
the control unit controls the heater unit so that the gas sensor chip has a predetermined temperature and detects a gas in the atmosphere based on information obtained using the gas detection material layers.

11. The gas sensing system according to claim 10,
wherein the number of the gas sensor chip and the number of the heater unit are each plural,
the heater units are placed in the gas sensor chips, respectively, and
the control unit controls the heater units so that the gas sensor chips have respectively different temperatures and detects a gas in the atmosphere based on information obtained using the gas detection material layers in each of the gas sensor chips.

12. A method of producing a gas sensor chip, comprising:
a step of forming an impurity layer on a semiconductor substrate;
a step of forming a first gas detection material layer including a catalyst metal layer on the semiconductor substrate; and
a step of forming a second gas detection material layer containing a catalyst metal layer at a position different from that of the first gas detection material layer, on the semiconductor substrate,
wherein the first gas detection material layer is different from the second gas detection material layer in at least one of a type, a film thickness, and a crystal particle size of a material constituting the gas detection material layers.

13. The method of producing a gas sensor chip according to claim 12,
wherein at least one the step of forming the first gas detection material layer and the step of forming the second gas detection material layer comprises a step of forming a metal oxide layer.

14. The method of producing a gas sensor chip according to claim 12,
wherein the step of forming the first gas detection material layer and the step of forming the second gas detection material layer are performed while being divided in plural film formation steps with a mask.

15. The method of producing a gas sensor chip according to claim 13,
wherein the step of forming the metal oxide layer comprises:
a step of forming a metal layer, and
a step of oxidizing the metal layer thus formed.
